SEQUENCE LISTING (2 sequences)

SEQ ID NO: 1 - protein, 389 aa, Homo sapiens [sequence omitted]

SEQ ID NO: 2 - protein, 418 aa, Homo sapiens
MRLSAGPDAGPSGNSSPWWPLATGAGNTSREAEALGEGNGPPRDVRNEELAKLEIAVLAVTFAVAVLGNSSVLLALHRTPRKTSRMHLFIRHLSLADLAVAFFQVLPQMCWDITYRFRGPDWLCRVVKHLQVFGMFASAYMLVVMTADRYIAVCHPLKTLQQPARRSRLMIAAAWVLSFVLSTPQYFVFSMIEVNNVTKARDCWATFIQPWGSRAYVTWMTGGIFVAPVVILGTCYGFICYNIWCNVRGKTASRQSKGAEQAGVAFQKGFLLAPCVSSVKSISRAKIRTVKMTFVIVTAYIVCWAPFFIIQMWSVWDPMSVWTESENPTITITALLGSLNSCCNPWIYMFFSGHLLQDCVQSFPCCQNMKEKFNKEDTDSMSRRQTFYSNNRSPTNSTGMWKDSPKSSKSIKFIPVST

(19) United States
(12) Patent Application Publication
(10) Pub. No.: US 2015/0072995 A1
(43) Pub. Date: Mar. 12, 2015

(54) TREATMENT OF MALE SEXUAL DYSFUNCTION

(71) Applicant: Ixchelsis Limited, Sandwich (GB)

(72) Inventors: Alasdair Mark Naylor, Sandwich (GB); Rachel Jane Russell, Sandwich (GB); Stephen Derek Albert Street, Sandwich (GB); Kim-Wah Tang, Sandwich (GB); Pieter Hadewijn Van Der Graaf, Sandwich (GB); Christopher Peter Wayman, Sandwich (GB)

(73) Assignee: Ixchelsis Limited, Sandwich (GB)

(21) Appl. No.: 14/495,537

(22) Filed: Sep. 24, 2014

Related U.S. Application Data
(63) Continuation of application No. 13/742,613, filed on Jan. 16, 2013, now abandoned.
Application No. 13/742,613 is a continuation of application No. 12/987,345, filed on Jan. 10, 2011, now abandoned.
Application No. 12/987,345 is a continuation of application No. 12/339,549, filed on Dec. 19, 2008, now abandoned.
Application No. 12/339,549 is a continuation of application No. 10/350,924, filed on Jan. 24, 2003, now abandoned.
(60) Provisional application No. 60/357,445, filed on Feb. 14, 2002.

(30) Foreign Application Priority Data
Jan. 31, 2002 (GB) 0202282.0

(51) Int. Cl. A61K 31/495; A61K 45/06

(52) U.S. Cl. CPC A61K 31/495; A61K 45/06
USPC 514/255.03

(57) ABSTRACT

A composition comprising a selective oxytocin antagonist for use in the treatment and/or prevention of a male ejaculatory disorder; which selective oxytocin antagonist is optionally admixed with a pharmaceutically acceptable carrier, diluent or excipient.

### CONTINUING APPLICATION DATA

This application is a continuation of U.S. Ser. No. 13/742,613, filed Jan. 16, 2013, which is continuation of U.S. Ser. No. 12/987,345, filed Jan. 10, 2011, now abandoned, which is a continuation of U.S. Ser. No. 12/339,549, filed Dec. 19, 2008, now abandoned, which is a continuation of U.S. Ser. No. 10/350,924, filed Jan. 24, 2003, now abandoned, which claims the benefit of U.S. Provisional Ser. No. 60/357,445, filed Feb. 14, 2002, each of which is incorporated by reference herein.

### FIELD OF INVENTION

The present invention relates to a compound and a pharmaceutical that is useful for the treatment and/or prevention of male sexual dysfunction, in particular ejaculatory disorders, such as premature ejaculation.

The present invention also relates to a method of prevention and/or treatment of male sexual dysfunction, in particular ejaculatory disorders, such as premature ejaculation.

The present invention also relates to assays to screen for the compounds useful in the treatment of male sexual dysfunction, in particular ejaculatory disorders, such as premature ejaculation.

### BACKGROUND TO THE INVENTION

### Male Sexual Dysfunction

Sexual dysfunction (SD) is a significant clinical problem which can affect both males and females. The causes of SD may be both organic as well as psychological. Organic aspects of SD are typically caused by underlying vascular diseases, such as those associated with hypertension or diabetes mellitus, by prescription medication and/or by psychiatric disease such as depression. Physiological factors include fear, performance anxiety and interpersonal conflict.

SD impairs sexual performance, diminishes self-esteem and disrupts personal relationships thereby inducing personal distress. In the clinic, SD disorders have been divided into female sexual dysfunction (FSD) disorders and male sexual dysfunction (MSD) disorders (Melman et al 1999 J. Urology 161 5-11). FSD is best defined as the difficulty or inability of a woman to find satisfaction in sexual expression. Male sexual-dysfunction (MSD) is generally associated with either erectile dysfunction, also known as male erectile dysfunction (MED) and/or ejaculatory disorders such as premature ejaculation, anorgasmia (unable to achieve orgasm) or desire disorders such as hypoactive sexual desire disorder (lack of interest in sex).

### Premature Ejaculation (PE)

PE is a relatively common sexual dysfunction in men. It has been defined in several different ways but the most widely accepted is the Diagnostic and Statistical Manual of Mental Disorders IV one which states:
“PE is a lifelong persistent or recurrent ejaculation with minimal sexual stimulation before, upon or shortly after penetration and before the patient wishes it. The clinician must take into account factors that affect duration of the excitement phase, such as age, novelty of the sexual partner or stimulation, and frequency of sexual activity. The disturbance causes marked distress of interpersonal difficulty.”

The International Classification of Diseases 10 definition states:
“There is an inability to delay ejaculation sufficiently to enjoy lovemaking, manifest as either of the following: (1) occurrence of ejaculation before or very soon after the beginning of intercourse (if a time limit is required: before or within 15 seconds of the beginning of intercourse); (2) ejaculation occurs in the absence of sufficient erection to make intercourse possible. The problem is not the result of prolonged abstinence from sexual activity”

Other definitions which have been used include classification on the following criteria:
Related to partner's orgasm
Duration between penetration and ejaculation
Number of thrust and capacity for voluntary control

Psychological factors may be involved in PE, with relationship problems, anxiety, depression, prior sexual failure all playing a role.

The estimated prevalence of PE is about 22-38% of the male population. Unlike male erectile dysfunction (MED), PE has no definite correlation with age. Taking an average prevalence of 30%, that would make an estimated 24 million sufferers in the US (males ages 18-65 was 80 million in 1995). There is little data on prevalence by severity. It is estimated that the operational definition of PE may apply to 5-10% of men, however, less than 0.2% present for treatment. The availability of an orally effective therapy is very likely to alter this situation.

Urologists currently form the bulk (59%) of physicians treating PE. GP's form 33% of doctors treating the condition. Sex therapists, behavioural therapists and counselors also treat patients with PE. Experts estimate that 50% of presenters do so because of the impact the condition has on the relationship with the partner. Stress, relationship difficulties and/or effect on quality of life are the key triggers for sufferers to seek treatment for PE.

Ejaculation is dependent on the sympathetic and parasympathetic nervous systems. Efferent impulses via the sympathetic nervous system to the vas deferens and the epididymis produce smooth muscle contraction, moving sperm into the posterior urethra. Similar contractions of the seminal vesicles, prostatic glands and the bulbouretheral glands increase the volume and fluid content of semen. Expulsion of semen is mediated by efferent impulses originating from the nucleus of Onuf in the spinal cord, which pass via the parasympathetic nervous system and cause rhythmic contractions of the bulbocavernous, ischiocavernous and pelvic floor muscles. Cortical control of ejaculation is still under debate in humans. In the rat the medial pre-optic area and the paraventricular nucleus of the hypothalamus seem to be involved in ejaculation.

There are at present no approved drugs available for treating PE. The most commonly off-label prescribed medications are the anti-depressants (for example clomipramine) and the selective serotonin re-uptake inhibitors (for example paroxetine and sertraline). These drugs are often not well accepted by patients because they are regarded as anti-depressants. They are used ‘off-label’, and though effective when used as required (i.e. ‘prn’), due to their long pharmacokinetic Tₘₐₓ (time to maximum drug concentration in plasma following oral administration of the drug) they are likely to have a slow onset of action. Side-effects common to this class of drugs can be seen when used chronically. Behavioural therapy has been the other management tool but has not been very efficacious and has a high drop-out and relapse rate. New, more efficient therapies are required.

Thus, it is desirable to find new ways of treating male sexual dysfunction, in particular ejaculatory disorders, such as premature ejaculation.

### SUMMARY ASPECTS

A seminal finding of the present invention is that by administering a selective oxytocin antagonist, an increase in latency to ejaculation can be achieved. Thus, it has been shown that by use of a selective oxytocin antagonist, treatment of ejaculatory disorders, in particular premature ejaculation, can be effected. This may be achieved by increasing ejaculatory latency, preferably by restoring ejaculatory latency to near normal levels.

In particular, use of a selective oxytocin antagonist results in the treatment of ejaculatory disorders, in particular premature ejaculation, whilst maintaining erectogenic mechanisms, in particular penile erection.

The treatment of ejaculatory disorders, in particular premature ejaculation, with a selective oxytocin antagonist allows the treatment thereof whilst maintaining the patient's sexual drive. The term “sexual drive” as used herein means libido or sexual desire.

Thus, compounds according to the present invention preferably comprise the unexpected advantage of maintaining erectogenic mechanisms, in particular penile erection, and/or sexual drive, as compared with known non-selective oxytocin antagonists.

### The Role of Oxytocin in Sexual Behaviour

Ejaculation comprises two separate components—emission and ejaculation. Emission is the deposition of seminal fluid and sperm from the distal epididymis, vas deferens, seminal vesicles and prostrate into the prostatic urethra. Subsequent to this deposition is the forcible expulsion of the seminal contents from the urethral meatus. Ejaculation is distinct from orgasm, which is purely a cerebral event. Often the two processes are coincidental.

A pulse of oxytocin in peripheral serum accompanies ejaculation in mammals. In man oxytocin but not vasopressin plasma concentrations are significantly raised at or around ejaculation. Oxytocin does not induce ejaculation itself; this process is 100% under nervous control via α1-adrenoceptor/sympathetic nerves originating from the lumbar region of the spinal cord. The systemic pulse of oxytocin may have a direct role in the peripheral ejaculatory response. It could serve to modulate the contraction of ducts and glandular lobules throughout the male genital tract, thus influencing the fluid volume of different ejaculate components for example. Oxytocin released centrally into the brain could influence sexual behaviour, subjective appreciation of arousal (orgasm) and latency to subsequent ejaculation. The occurrence of ejaculation in males is critically dependent on tactile stimulation of the external genitalia.

It is well documented that the levels of circulating oxytocin increase during sexual stimulation and arousal, and peak during orgasm in both men and women.

Murphy et al. (Acta. Anat. Basel 128: 76-79 [1987]) measured the plasma oxytocin and arginine vasopressin (AVP) concentrations in men during sexual arousal and ejaculation and found that plasma AVP but not oxytocin significantly increased during sexual arousal. However, at ejaculation, mean plasma oxytocin rose about five-fold and fell back to basal concentrations within 30 minutes, while AVP had already returned to basal levels at the time of ejaculation and remained stable thereafter.

As detailed in Gimpl and Fahrenholz (Physiological Reviews Vol. 81: No. 2. April 2001 pp 629-683), oxytocin has been found to be one of the most potent agents to induce penile erection in rats, rabbits and monkeys. In addition, central administration of oxytocin is claimed to reduce the latency to achieve ejaculation and to shorten the post-ejaculatory interval. Likewise, Meston et al (Arch. Gen Psychiatry, Vol. 57, November 2000) states that in male animals, oxytocin facilitates penile erections when injected into specific areas of the brain (i.e. periventricular nucleus of the hypothalamus) and shortens the ejaculation latency and post-ejaculation interval when injected either centrally or peripherally.

It has been well documented within the art that the administration of the oxytocin receptor agonist, vasotocin, significantly reduces non-contact penile erections (see, for example, Melis et al (Neuroscience Letters 265 (1999) 171-174). In addition, intracerebroventricular (ICV) injection of the oxytocin antagonist vasotocin was shown in Argiolas et al (European Journal of Pharmacology 149 (1988) 389-392) to impair sexual performance in experienced male rats in the presence of a receptive female, with the abolishment of ejaculation (probably caused by a decreased intromission frequency). The decrease in intromission frequency was thought to reflect a decreased capacity of the animals to achieve penile erection, as the oxytocin antagonist was found to prevent penile erection.

Although in Gimple and Farenholz (supra) and Meston et al. (supra) it was suggested that oxytocin reduces the latency to achieve ejaculation, alternative studies have shown oxytocin to have no effect on ejaculatory latency. For example, in Stoneham et al. (J. Endocrinology 107: 97-106, 1985) it is shown that intravenous infusion of oxytocin in rats dose dependently reduced the number of intromissions made before ejaculation but had no effect on ejaculatory latency. Also, infusion of oxytocin into the 3^{rd} ventricle increased the latencies to the first mount and intromission, and lengthened post ejaculatory refractory periods, but had no effect on ejaculatory latency (Stoneham et al supra).

In addition, studies have shown that abolishing the increase in oxytocin at ejaculation made no difference to the time taken to achieve arousal or orgasm. In Murphy et al. (J. of Clinical Endocrinology and Metabolism, Vol. 71, No. 4 (1990) p 1056-1058) the opioid antagonist naloxone, was shown to have no effect on ejaculation in human volunteers, even though the serum oxytocin pulse typically observed at ejaculation was eliminated. In Ackerman et al. (Physiol Behav 63: 49-53 [1997]) N-methyl-D-aspartic acid lesions, which destroy parvocellular PVN neurons while leaving magnocellular neurons intact, reduced oxytocin-immunoreactive fibres in the lower lumbar spinal cord (L5-L6). This reduction was associated with a significant decrease in seminal emission at the time of ejaculation, but mount, intromission and ejaculatory latencies were unaffected.

### Detailed Aspects

In one aspect the present invention relates to a composition or a pharmaceutical composition comprising a selective oxytocin antagonist compound for use in the treatment and/or prevention of a male ejaculatory disorder, in particular premature ejaculation. In the pharmaceutical composition the selective oxytocin antagonist is optionally admixed with a pharmaceutically acceptable carrier, diluent or excipient. Here, the composition (like any of the other compositions mentioned herein) may be packaged for subsequent use in the treatment of a male ejaculatory disorder, in particular premature ejaculation.

In another aspect the present invention relates to a composition or a pharmaceutical composition comprising a selective oxytocin antagonist compound for use in the treatment and/or prevention of a male ejaculatory disorder, in particular premature ejaculation whilst maintaining erectogenic mechanisms, in particular penile erection, and/or sexual drive; wherein said composition is optionally admixed with a pharmaceutically acceptable carrier, diluent or excipient.

In another aspect, the present invention relates to the use of a selective oxytocin antagonist in the manufacture of a medicament (such as a pharmaceutical composition) for use in the treatment of a male ejaculatory disorder, in particular premature ejaculation.

In another aspect, the present invention relates to the use of a selective oxytocin antagonist in the manufacture of a medicament (such as a pharmaceutical composition) for use in the treatment of a male ejaculatory disorder, in particular premature ejaculation, whilst maintaining erectogenic mechanisms, in particular penile erection, and/or sexual drive.

In another aspect, the present invention relates to the use of a selective oxytocin antagonist in the preparation of a medicament (such as a pharmaceutical composition) for use in the treatment of a male ejaculatory disorder, in particular premature ejaculation.

In another aspect, the present invention relates to the use of a selective oxytocin antagonist in the preparation of a medicament (such as a pharmaceutical composition) for use in the treatment of a male ejaculatory disorder, in particular premature ejaculation whilst maintaining erectogenic mechanisms, in particular penile erection, and/or sexual drive.

In one aspect, the present invention relates to a method of treating and/or preventing a male ejaculatory disorder, in particular premature ejaculation, in a human or animal which method comprises administering to an individual an effective amount of a selective oxytocin antagonist, wherein said selective oxytocin antagonist is optionally admixed with a pharmaceutically acceptable carrier, diluent or excipient.

In one aspect, the present invention relates to a method of treating and/or preventing a male ejaculatory disorder, in particular premature ejaculation, whilst maintaining erectogenic mechanisms, in particular penile erection, and/or sexual drive, in a human or animal which method comprises administering to an individual an effective amount of a selective oxytocin antagonist, wherein said selective oxytocin antagonist is optionally admixed with a pharmaceutically acceptable carrier, diluent or excipient.

There is further provided a pharmaceutical pack comprising one or more compartments wherein at least one compartment comprises one or more of a selective oxytocin antagonist.

The present invention further provides a process of preparation of a pharmaceutical composition according to the present invention, said process comprising admixing one or more selective oxytocin antagonists with a pharmaceutically acceptable diluent, excipient or carrier.

In a further aspect, the present invention relates to an assay method for identifying an agent (hereinafter referred to as a selective oxytocin antagonist) that can be used to treat or prevent a male ejaculatory disorder, in particular premature ejaculation, the assay comprising: determining whether a test agent can directly delay the endogenous ejaculatory process; wherein said delay is defined as an increase in and/or restoration of ejaculatory latency (i.e. time taken from first intromission to ejaculation) in the presence of a test agent as defined herein; such potentiation by a test agent is indicative that the test agent may be useful in the treatment and/or prevention of a male ejaculatory disorder, in particular premature ejaculation, and wherein said test agent is a selective oxytocin antagonist. Preferably, the agent has no, or substantially no, effect on penile erection. That is to say, preferably, the agent does not adversely affect penile erection; however, the agent may enhance endogenous penile erection.

In a further aspect, the present invention relates to a process comprising the steps of: [0044] (a) performing the assay method according to the present invention; [0045] (b) identifying one or more agents capable of increasing and/or restoring ejaculatory latency; and [0046] (c) preparing a quantity of those one or more identified agents; and wherein said agent is a selective oxytocin antagonist.

With this aspect, the agent identified in step (b) may be modified so as to maximise, for example, activity and then step (a) may be repeated. These steps may be repeated until the desired activity or pharmacokinetic profile has been achieved.

Thus, in a further aspect, the present invention relates to a process comprising the steps of: (a1) performing the assay according to the present invention; (b1) identifying one or more agents that can directly increase and/or restore ejaculatory latency; (b2) modifying one or more of said identified agents; (a2) optionally repeating step (a1); and (c) preparing a quantity of those one or more identified agents (i.e. those that have been modified); and wherein said agent is a selective oxytocin antagonist.

In a further aspect, the present invention relates to a process comprising the steps of: [0050] (i) performing the assay method according to the present invention; [0051] (ii) identifying one or more agents capable of increasing and/or restoring ejaculatory latency; [0052] (iii) testing identified agents for their effect on penile erection in test animals, such as anaesthetised rodents; [0053] (iv) selecting agents with no, or substantially no, effect on penile erection; and [0054] (v) preparing a quantity of those one or more selected agents; and wherein said agent is a selective oxytocin antagonist.

With this aspect, the agent identified in step (ii) may be modified so as to maximise, for example, activity and then step (i) may be repeated. These steps may be repeated until the desired activity or pharmacokinetic profile has been achieved.

In a further aspect, the present invention relates to a diagnostic method, the method comprising isolating one or more samples from a male during sexual stimulation at successive time intervals, i.e. 15 seconds, 30 seconds, 1 minute, 2 minutes, 3 minutes, 4 minutes and 5 minutes following the commencement of sexual stimulation, determining whether the sample(s) contains an entity present at such a time and in such an amount as to cause a male ejaculatory disorder, preferably premature ejaculation; and wherein said entity can be modulated, in particular the time taken for the entity to appear and/or the peak in concentration can be delayed, to achieve a beneficial effect by use of an agent; and wherein said agent is a selective oxytocin antagonist. Preferably, the entity is oxytocin. The sexual stimulation may be caused by a penile vibratory stimulation device (FertiCare, Horsholm, Denmark), for example.

In a further aspect, the present invention relates to a diagnostic composition or kit comprising means for detecting an entity in one or more isolated male samples which sample(s) is taken at successive time intervals, i.e. 15 seconds, 30 seconds, 1 minute, 2 minutes, 3 minutes, 4 minutes and 5 minutes following the commencement of sexual stimulation, during sexual stimulation of said male; wherein the means can be used to determine whether the sample(s) contains the entity at such a time and in such an amount as to cause a male ejaculatory disorder, preferably premature ejaculation; and wherein said entity can be modulated, in particular the time taken for the entity to appear and/or the peak in concentration can be delayed, to achieve a beneficial effect by use of an agent; and wherein said agent is a selective oxytocin antagonist. Preferably, the entity is oxytocin. The sexual stimulation may be caused by a penile vibratory stimulation device (FertiCare, Horsholm, Denmark), for example.

In a further aspect, the present invention relates to an animal model used to identify agents capable of treating and/or preventing a male ejaculatory disorder, in particular premature ejaculation, said model comprising a male animal including means to measure ejaculation latency (i.e. time taken from first intromission to ejaculation) of said animal following introduction of a receptive female; and wherein said agent is a selective oxytocin antagonist.

The animal model may further comprise or be used in conjunction with an additional animal model comprising means to measure changes in penile erection. For example, a suitable additional model may be one comprising an anaesthetised male animal including means to measure changes in intracavernosal pressure and/or cavernosal blood flow of said animal following stimulation of the pelvic nerve thereof; and wherein said agent is a selective oxytocin antagonist.

In a further aspect, the present invention relates to an assay method for identifying an agent that can directly enhance the endogenous ejaculatory processes in order to treat or prevent ejaculatory disorders, in particular premature ejaculation, the assay method comprising: administering an agent to the animal model of the present invention; and measuring ejaculation latency (i.e. time taken from first intromission to ejaculation) of said animal following introduction of a receptive female; and wherein said agent is a selective oxytocin antagonist.

In a further aspect, the present invention relates to an assay method for identifying an agent that can directly enhance the endogenous ejaculatory process without affecting penile erection and/or sexual drive in order to treat or prevent ejaculatory disorders, in particular premature ejaculation, the assay method comprising: administering an agent to the animal model of the present invention; and measuring the change in the endogenous ejaculatory process; wherein said change is defined as ejaculation latency (i.e. time taken from first intromission to ejaculation) of said animal following introduction of a receptive female; measuring penile erection and/or sexual drive in the animal model to ensure no or substantially no change therein; and wherein said agent is a selective oxytocin antagonist.

For ease of reference, these and further aspects of the present invention are now discussed under appropriate section headings. However, the teachings under each section are not necessarily limited to each particular section.

The terms “selective oxytocin antagonist” and “selective oxytocin receptor antagonist” are interchangeable and mean an oxytocin receptor antagonist which is selective towards oxytocin receptors as compared with vasopressin, in particular V1a, receptors.

The term “ejaculatory latency” as used herein means the time taken from first intromission to ejaculation. The term “restoration of ejaculatory latency” as used herein means that the time take from first intromission to ejaculation is modified, preferably increased. Preferably, the time taken from intromission to ejaculation is modified (preferably increased) to near normal levels. Typically, a person suffering from premature ejaculation ejaculates within 30 seconds of the beginning of intercourse (i.e. from first intromission) and often within 15 seconds of the beginning of intercourse (i.e. from first intromission). In a preferred aspect of the present invention ejaculatory latency is increased to at least above 30 seconds, preferably to at least above 60 seconds, more preferably to at least above 2 minutes, more preferably to at least above 5 minutes, more preferably to at least above 10 minutes. Suitably, ejaculatory latency may be restored such that the time taken from intromission to ejaculation is sufficiently delayed to allow for partner satisfaction.

The term “sexual drive” as used herein means libido or sexual desire.

The term “intromission” as used herein means vaginal penetration by the penis.

### Preferable Aspects

In one embodiment, preferably the agent for use according to the present invention is for oral administration.

In another embodiment, the agent for use according to the present invention may be for topical administration or intranasal administration.

Preferably, the agent according to the present invention is for use in the treatment and/or prevention of premature ejaculation.

Preferably, the selective oxytocin antagonist is at least 20-fold selective for an oxytocin receptor as compared with a vasopressin receptor, in particular a V1a receptor.

Preferably, the selective oxytocin antagonist is at least 30-fold selective for an oxytocin receptor as compared with a vasopressin receptor, in particular a V1a receptor.

Preferably, the selective oxytocin antagonist is at least 50-fold selective for an oxytocin receptor as compared with a vasopressin receptor, in particular a V1a receptor.

Preferably, the selective oxytocin antagonist is at least 100-fold selective for an oxytocin receptor as compared with a vasopressin receptor, in particular a V1a receptor.

Preferably, the selective oxytocin antagonist is at least 200-fold selective for an oxytocin receptor as compared with a vasopressin receptor, in particular a V1a receptor.

Preferably, the selective oxytocin antagonist is at least 250-fold selective for an oxytocin receptor as compared with a vasopressin receptor, in particular a V1a receptor.

The present invention also encompasses administration of the agent of the present invention before and/or during sexual arousal/stimulation.

Thus, for some aspects of the present invention it is highly desirable that there is a sexual arousal/stimulation step.

Here, “sexual arousal/stimulation” may be one or more of a visual arousal/stimulation, a physical arousal/stimulation, an auditory arousal/stimulation or a thought arousal/stimulation.

Thus, preferably the agents of the present invention are delivered before or during sexual arousal/stimulation, particularly when those agents are for oral delivery.

### Preferred Aspects

The present invention provides the following (numbered) preferred aspects:
1. A composition comprising a selective oxytocin antagonist for use in the treatment or prevention of a male ejaculatory disorder; which selective oxytocin antagonist is optionally admixed with a pharmaceutically acceptable carrier, diluent or excipient.
2. A composition according to aspect 1 wherein the male ejaculatory disorder is a premature ejaculation.
3. A composition according to aspect 1 or aspect 2 wherein the selective oxytocin antagonist is at least 20-fold selective for an oxytocin receptor as compared with a vasopressin receptor.
4. A composition according to aspect 3 wherein the vasopressin receptor is a V1a receptor.
5. The use of a selective oxytocin antagonist in the manufacture of a medicament for use in the treatment of a male ejaculatory disorder.
6. The use according to aspect 5 wherein the male ejaculatory disorder is premature ejaculation.
7. The use according to aspect 5 or aspect 6 wherein the selective oxytocin antagonist is at least 20-fold selective for an oxytocin receptor as compared with a vasopressin receptor.
8. The use according to aspect 7 wherein the vasopressin receptor is a V1a receptor.
9. The use according to any one of aspects 5-8 wherein said selective oxytocin antagonist is administered before and/or during sexual arousal.
10. The use according to any one of aspects 5-9 wherein said selective oxytocin antagonist is administered by mouth.
11. A method of treating or preventing a male ejaculatory disorder in a human or animal which method comprises administering to an individual an effective amount of a selective oxytocin antagonist; wherein said selective oxytocin antagonist is optionally admixed with a pharmaceutically acceptable carrier, diluent or excipient.
12. A method according to aspect 11 wherein the male ejaculatory disorder is premature ejaculation.
13. A method according to aspect 11 or aspect 12 wherein said selective oxytocin antagonist is at least 20-fold selective for an oxytocin receptor as compared with a vasopressin receptor.
14. A method according to aspect 13 wherein the vasopressin receptor is a V1a receptor.
15. The method according to any one of aspects 11-14 wherein said selective oxytocin antagonist is administered before and/or during sexual arousal.
16. The method according to any one of aspects 11-15 wherein the medicament is administered by mouth.
17. A pharmaceutical pack comprising one or more compartments wherein at least one compartment comprises one or more of a selective oxytocin antagonist.
18. A pharmaceutical pack according to aspect 17 wherein said selective oxytocin antagonist is at least 20-fold selective for an oxytocin receptor as compared with a vasopressin receptor.
19. A pharmaceutical pack according to aspect 18 wherein said vasopressin receptor is a V1a receptor.
20. A process of preparation of a pharmaceutical composition, said process comprising admixing one or more selective oxytocin antagonists with a pharmaceutically acceptable diluent, excipient or carrier.
21. A process according to aspect 20 wherein the selective oxytocin antagonist is at least 20-fold selective for an oxytocin receptor as compared with a vasopressin receptor.
22. A process according to aspect 21 wherein the vasopressin receptor is a V1a receptor.
23. An assay method for identifying an agent that can be used to treat and/or prevent a male ejaculatory disorder, the assay comprising: determining whether a test agent can directly enhance the endogenous ejaculatory process; wherein said enhancement is defined as an increase in and/or restoration of ejaculatory latency in the presence of a test agent as defined herein; such potentiation by a test agent is indicative that the test agent may be useful in the treatment or prevention of a male ejaculatory disorder, and wherein said test agent is a selective oxytocin antagonist.
24. An assay according to aspect 23 wherein said male ejaculatory disorder is premature ejaculation.
25. An assay according to aspect 23 or aspect 24 wherein the selective oxytocin antagonist is at least 20-fold selective for an oxytocin receptor as compared with a vasopressin receptor.
26. An assay according to aspect 25 wherein the vasopressin receptor is a V1a receptor.
27. An agent identified by the assay method according to any one of aspects 23 to 26.
28. An agent according to aspect 27 for use in treating or preventing a male ejaculatory disorder.
29. An agent according to aspect 28 wherein said male ejaculatory disorder is premature ejaculation.
30. A medicament for oral administration to treat a male ejaculatory disorder, wherein the medicament comprises the agent according to aspect 27.
31. A medicament according to aspect 30 wherein said male ejaculatory to disorder is premature ejaculation.
32. A medicament according to aspect 30 or aspect 31 wherein said medicament is administered before and/or during sexual arousal.
33. A medicament according to any one of aspects 30-32 wherein the medicament is administered by mouth.
34. A process comprising the steps of: (a) performing the assay method of any one of aspects 23-26; (b) identifying one or more agents capable of increasing and/or restoring ejaculatory latency; and (c) preparing a quantity of those one or more identified agents; and wherein said agent is a selective oxytocin antagonist.
35. A process according to aspect 34 wherein the selective oxytocin antagonist is at least 20-fold selective for an oxytocin receptor as compared with a vasopressin receptor.
36. A process according to aspect 35 wherein the vasopressin receptor is a V1a receptor.
37. An animal model for identifying an agent capable of treating or preventing a male ejaculatory disorder, said model comprising a male animal including means to measure ejaculation latency of said animal following introduction of a receptive female; and wherein said agent is a selective oxytocin antagonist.
38. An animal model according to aspect 37 wherein said male ejaculatory disorder is premature ejaculation.
39. An animal model according to aspect 37 or aspect 38 wherein the selective oxytocin antagonist is at least 20-fold selective for an oxytocin receptor as compared with a vasopressin receptor.
40. An animal model according to aspect 39 wherein the vasopressin receptor is a V1a receptor.
41. An assay method for identifying an agent that can directly enhance the endogenous ejaculatory processes in order to treat or prevent ejaculatory disorders, the assay method comprising: administering an agent to the animal model of any one of aspects 37 to 40; and measuring ejaculation latency of said animal following introduction of a receptive female; and wherein said agent is a selective oxytocin antagonist.
42. The use of a combination consisting of one or more selective oxytocin antagonists and one or more of the following auxiliary active agents in the manufacture/preparation of a medicament for the treatment and/or prevention of a male ejaculatory disorders:
   i) A PDE inhibitor, more particularly a PDE 5 inhibitor, said inhibitors preferably having an IC50 against the respective enzyme of less than 100 nM;
   ii) A serotonin receptor agonist or modulator, more particularly agonists or modulators for 5HT2C, 5HT1B and/or 5HT1D receptors, including anpirtoline;
   iii) A serotonin receptor antagonist or modulator, more particularly antagonists or modulators for 5HT1A, including NAD-299 (robalzotan) and WAY-100635, and/or more particularly antagonists or modulators for 5HT3 receptors, including batanopirde, granisetron, ondansetron, tropistron and MDL-73147EF;
   iv) An antidepressant, in particular i) a selective serotonin re-uptake inhibitor (SSRI), including sertraline, fluoxetine, fluvoxamine, paroxetine, citalopram, venlafaxine, mirtazapine, nefazodone and trazodone; ii) a tricyclic antidepressant (TCA), including clomipramine, desapramine, imipramine, amitriptyline, doxepine, amoxapine, maprotiline, nortriptyline, protriptyline, trimipramine and buproprion; and iii) monoamine oxidase;
   v) An .alpha.-adrenergic receptor antagonist (also known as .alpha.-adrenergic blockers, .alpha-blockers or .alpha-receptor blockers); suitable .alpha.1-adrenergic receptor antagonists include: phentolamine, prazosin, phentolamine mesylate, trazodone, alfuzosin, indoramin, naftopidil, tamsulosin, phenoxybenzamine, rauwolfa alkaloids, Recordati 15/2739, SNAP 1069, SNAP 5089, RS17053, SL 89.0591, doxazosin, terazosin and abanoquil; suitable .alpha.2-adrenergic receptor antagonists include dibenamine, tolazoline, non-selective .alpha.-adrenergic receptor antagonists include dapiprazole; further .alpha.-adrenergic receptor antagonists are described in WO99/30697, U.S. Pat. No. 4,188,390, U.S. Pat. No. 4,026,894, U.S. Pat. No. 3,511,836, U.S. Pat. No. 4,315,007, U.S. Pat. No. 3,527,761, U.S. Pat. No. 3,997,666, U.S. Pat. No. 2,503,059, U.S. Pat. No. 4,703,063, U.S. Pat. No. 3,381,009, U.S. Pat. No. 4,252,721 and U.S. Pat. No. 2,599,000; [0128] vi) A rapid onset selective serotonin re-uptake inhibitor.
43. The use of a combination consisting of one or more selective oxytocin antagonists and one or more PDE inhibitors (PDEi's) in the manufacture/preparation of a medicament for the treatment or prevention of a male ejaculatory disorder.
44. The use according to aspect 42 or aspect 43 wherein said male ejaculatory disorder is premature ejaculation.
45. The use according to aspect 43 or aspect 44 wherein said PDEi is a PDE5 inhibitor (PDE5i).
46. The use according to any one of aspects 42-45 wherein the medicament is administered by mouth.
47. A pharmaceutical composition consisting of one or more selective oxytocin antagonists and one or more PDEi's, optionally admixed with a pharmaceutically acceptable carrier, diluent or excipient.
48. A pharmaceutical composition according to aspect 47 wherein said PDEi is a PDE5i. 49. A pharmaceutical composition according to any one of aspects 47 or 48 wherein the composition is administered by mouth.
50. The use of a pharmaceutical composition according to any one of aspects 47-49 in the preparation of a medicament for the treatment and/or prevention of ejaculatory disorders.

### Surprising and Unexpected Findings

The present invention demonstrates the surprising and unexpected findings that:
(a) administration of a selective oxytocin antagonist increases ejaculatory latency. Preferably, administration of a selective oxytocin antagonist restores ejaculatory latency, preferably to near normal levels;
(b) administration of a selective oxytocin antagonist unexpectedly increases ejaculatory latency without substantially inhibiting and/or adversely affecting erectogenic mechanisms, in particular penile erection. Preferably administration of a selective oxytocin antagonist restores ejaculatory latency, preferably to near normal levels, without substantially inhibiting and/or adversely affecting erectogenic mechanisms, in particular penile erection;
(c) administration of a selective oxytocin antagonist increases ejaculatory latency without substantially inhibiting and/or adversely affecting sexual drive. Preferably administration of a selective oxytocin antagonist restores ejaculatory latency, preferably to near normal levels, without substantially inhibiting and/or adversely affecting sexual drive.

### Advantages

The present invention is advantageous because:
(a) selectively inhibiting oxytocin receptors by use of a selective oxytocin antagonist results in the treatment of premature ejaculation
(b) selectively inhibiting oxytocin receptors by use of a selective oxytocin antagonist unexpectedly results in the treatment of premature ejaculation without substantially inhibiting and/or adversely affecting erectogenic mechanisms, in particular penile erection;
(c) selectively inhibiting oxytocin receptors by use of a selective oxytocin antagonist unexpectedly results in the treatment of premature ejaculation without substantially inhibiting and/or adversely affecting sexual drive.

### Patient Groups

Patients with ejaculatory disorders, in particular premature ejaculation, should benefit from treatment with a selective oxytocin antagonist.

Early investigations suggest the below mentioned ejaculatory disorder, in particular premature ejaculation, patient groups should benefit from treatment with a selective oxytocin antagonist (or a combination comprising a selective oxytocin antagonist as set out hereinafter). These patient groups include those suffering from one or more of the following: a neurological disorder, a physiological disorder, psychosexual skills deficit, a physical illness, a physical injury, pharmacological side effects, physchological distress and relationship distress.

### Oxytocin Receptors

As indicated above, the agent may be any suitable agent that can act as a selective oxytocin antagonist.

Background teachings on oxytocin receptors have been prepared by Victor A. McKusick, et al on http://www3.ncbi.nlm.nih.gov/Omim/searchomim.htm. The following text concerning oxytocin receptors has been extracted from that source:
“Kimura et al (1992: Nature 356: 526-529) reported the structure and expression of the human oxytocin receptor cDNA isolated by expression cloning. The encoded receptor was a 388-amino acid polypeptide with 7 transmembrane domains typical of G protein-coupled receptors. The oxytocin receptor, expressed in Xenopus oocytes, specifically responded to oxytocin and induced an inward membrane current. Messenger RNAs for the receptor were of 2 sizes, 3.6 kb in breast and 4.4 kb in ovary, endometrium, and myometrium. The mRNA level in myometrium was very high at term. Inoue et al. (1994 Biol. Chem. 269: 32451-32456) showed by Southern blots that the OXTR gene is present in single copy in the human genome. By fluorescence in situ hybridisation, they demonstrated that the gene is located on 3p26.2. The gene spans approximately 17 kb and contains 3 introns and 4 exons. Exons 1 and 2 correspond to the 5-prime noncoding region, followed by exons 3 and 4 encoding the amino acids of the receptor. Intron 3, which is the largest at 12 kb, separates the coding region immediately after the putative 6 transmembrane-spanning domain. The transcription start sites, demonstrated by primer extension analysis, lie 618 and 621 bp upstream of the methionine initiation codon. By PCR analysis of somatic cell hybrids and by fluorescence in situ hybridisation, Simmons et al. (1995 Genomics 26: 623-625) assigned the OXTR gene to 3p25.”

In Gimple and Fahrenholz (Physiological reviews Vol. 81, No. 2, April 2001) a detailed review of the receptor structure is presented. It is stated therein that, to date, in addition to the isolation and identification of a cDNA encoding the human oxytocin receptor (see Kimura et al [supra]) the oxytocin receptor encoding sequences from pig (Gorbulev at al., Eur. J. Biochem. 215, 1-7 1993), rat (Rozen et al., Proc. Natl. Acad. Sci. USA 92: 200-204 1995), sheep (Riley et al., J. Biol. Chem. 266: 21428-21433, 1991), bovine (Bathgate et al., DNA Cell Biol. 14: 1037-1048, 1995), mouse (Kubota et al., I Mol. Cell. Endocrinol 124: 25-32 1996) and rhesus monkey (Salvatore et al., J. Recept Signal Transduct Res. 18: 15-24, 1998) have also been identified.

### Oxtocin Receptors Sequence Data

Nucleotide sequences and amino acid sequences for human oxytocin receptors are available in the literature. By way of example only an amino acid sequence for a human oxytocin receptor is, presented in SEQ ID NO: 1.

### Selective Oxytocin Antagonists

Details of suitable assay systems for identifying and/or studying oxytocin antagonists are presented hereinafter in the section entitled “Oxytocin Antagonist Assay”.

An example of a suitable oxytocin antagonist is presented below:

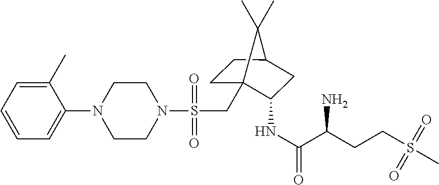 [SMILES: Cc1ccccc1N1CCN(S(=O)(=O)CC23CCC(C[C@@H]2NC(=O)[C@@H](N)CCS(C)(=O)=O)C3(C)C)CC1]

The synthesis of L-368,899 is taught in Williams et al., (1994) J. Med. Chem. 37, 565-571.

L-368,899 is a selective oxytocin antagonist. L-368,899 is over 20-fold selective for oxytocin receptors over vasopressin, in particular V1a, receptors. Preferably the selectivity is both binding and functional selectivity.

Certain known oxytocin antagonists, such as vasotocin for instance are sometimes referred to a being “selective oxytocin antagonists”. However, d(CH₂)₅Tyr(Me)-Orn⁸-vasotocin (hereinafter referred to as “vasotocin”) in only 2- to 3-fold selective for oxytocin receptors over vasopressin, in particular V1a, receptors. As such, vasotocin in a substantially non-selective oxytocin/vasopressin antagonist and does not fall within the scope of the term “selective oxytocin antagonist” according to the present invention. Preferably, the selective oxytocin antagonist according to the present invention is at least 20-fold selective for oxytocin receptors over vasopressin, in particular V1a, receptors.

### Oxytocin Antagonist Binding Assay and Vasopression V1a Binding Assay

### A. Oxytocin Receptor Ligand Binding IC50 Assay

### i) Buffers

| | |
|---|---|
| Cell Growth Medium | Hams F12 Nutrient Mix |
| | 10% FCS |
| | 2 mM L-Glutamine |
| | 400 μg/ml G418 |
| | 15 mM HEPES |
| Membrane Prep Buffer | 50 mM Tris-HCl, pH 7.8 |
| | 10 mM Mg Cl₂ |
| Protease Inhibitors | Freezing Buffer 50 mM Tris-HCl, pH 7.8 |
| | 10 mM Mg Cl₂ |
| | 20% Glycerol |
| Assay Medium | 50 mM Tris-HCl, pH 7.8 10 |
| | mM Mg Cl₂ |
| | 0.25% BSA |
| Max. | 0.5 μM (arg⁸)-vasotocin |
| | made in 2.5% DMSO/50 mM Tris- HCL, |
| | pH 7.8, 10 mM MgCl₂ |
| Min. | 2.5% DMSO/50 mM Tris- HCL, pH 7.8, |
| | 10 mM MgCl₂ |

### ii) Compound Dilution (Final Concentration of 10 μM in the Assay)

a) HTA stock compounds at 4 mM in 100% DMSO
b) Dilute compounds to 200 μM in dH₂O.
c) Further dilute compounds to 100 μM in 100 mM Tris-HCl, pH 7.8, 20 mM MgCl₂. This gives final concentrations of 2.5% DMSO, 50 mM Tris-HCl, pH 7.8, 10 mM MgCl₂.
d) Using the diluted stock, prepare 1:2 dilutions over 10 points in 50 mM Tris-HCl, pH 7.8, 10 mM MgCl₂, 2.5% DMSO with the TECAN Genesis.
e) Dispense 10 μl of the compound into a 384 well Optiplate according to the plate layout required for analysis by ECADA leaving space for the standard (arg⁸)-vasotocin IC50. These plates can be stored at 4° C.
f) On the day of the assay, add 10 μl of Max. to the +wells and 10 μl of Min. to the -wells, and a 1:2 serial dilution over 10 points in duplicate of the (arg⁸)-vasotocin with a top concentration of 100 nM (20 nM final).
   iii) Maintenance of the Oxytocin Receptor—CHO Cells
The cell line is routinely maintained as a continuous culture in 50 ml growth medium in 225 cm² flasks.
Cells are passaged by removing the medium from the monolayer, washing with PBS and incubating with Trypsin until cells show signs of dissociation. After knocking the cells from the bottom of the flask, cells are resuspended in growth medium and seeded into 225 cm² flasks at a concentration of 8×10⁵ cells/flask.

### iv) Growth of Cells in Roller Bottles

Cells are seeded into 10×850 cm² roller bottles at a density of 6×10⁶ cells/bottle and are allowed to reach near confluence.
Cells are removed from the bottles using trypsin, as described above, and the cells are seeded into 100× roller bottles (i.e. 1:10 split ratio).
Cells are again allowed to reach near confluence before removing the growth medium, adding 40 ml PBS/bottle and harvesting by scraping using the CellMate. The cell suspension is then centrifuged at 2000 rpm, washed in PBS, centrifuged again and pellets are frozen in aliquots at −80° C.

### v) Membrane Preparations

Cell pellets are retrieved from the freezer, thawed on ice and resuspended in 3 ml of membrane prep buffer per ml packed cell volume.
The suspension is then homogenised using a mechanical homogeniser for several bursts of 5 secs on ice before centrifuging at 25,000×g for 30 mins.
After resuspending the pellet in 1 ml of freezing buffer per 1 ml of the original packed cell volume the suspension is briefly homogenised to remove small lumps. Protein concentrations are then measured and the membrane suspension is finally frozen in aliquots at a minimum of 5 mg/ml at −80° C.

### vi) Assay

Membranes are thawed on ice before diluting to 1 mg/ml in assay buffer. SPA beads are resuspended at 50 mg/ml in assay buffer. From these concentrations, beads are pre-coupled with membranes by incubating 30 μg of protein per mg of bead on a top-to-tail shaker for 2 hours at 4° C. The bead/membranes are then centrifuged at 2000 rpm for 10 mins and the pellet is resuspended at 3 mg/ml.
All manipulations of the ¹²⁵I-OVTA are carried out using tips that have been silanised using SigmaCote. All bottles and tubes are also silanised. The ¹²⁵I-OVTA is diluted in 1 ml assay buffer per 50 μCi of lyophilised ligand. A 5 μl sample is then counted in duplicate using liquid scintillation counting (protocol 61 on Wallac Counter) and the concentration of the ligand is calculated (see example below). This is to overcome any loss of ligand due to stickiness. Using the measured concentration, the ¹²⁵I-OVTA is diluted to 0.3 nM in assay buffer.
   Example:
   If 5 μl gives 500000 dpm and the specific activity of the ligand is 2200 Ci/mmol then:
   Concentration=5000001(2.2×2200×5) nM
20 μl of the bead/membrane preparation is added to the prepared Optiplates using the Multi-drop. The bead/membrane preparation is kept in suspension using a stirring flask. 20 μl of the ¹²⁵I-OVTA is then added to each well of the Optiplate using the Multi-drop. Following a 4 hour incubation at room temperature, the plates are counted using the TopCount NXT for 30 s/well.

### B. Vasopressin VIA Receptor Binding Assay

i) Materials

| | |
|---|---|
| Human cloned vasopressin V1a receptor | Protein/cell sciences |
| in CHO cells | |
| HEPES | Sigma (H7523) |
| Magnesium Chloride (MgCl.sub.2) | Sigma (M2670) |
| Bovine serum albumin (BSA) | Sigma (A6003) |
| Glycerol | Sigma (G5150) |
| Protease Inhibitor Cocktail Tablets | Roche (1697498) |
| Pierce BCA Protein Assay Reagent | Pierce (23225) |
| 8-Arg[phenylalanyl-3,4,5-³H]-vasopressin(³H-AVP) | NEN (NET800) |
| d(CH2)5Tyr(Me)AVP β-mercapto-β,β- | Sigma (V2255) |
| cyclopentamethylene propionyl,O-Me-Tyr²,Arg⁸]- | |
| vasopressin βMCPVP) | |
| Dimethylsulphoxide | Stores (W34) |
| 96 well polypropylene blocks | Stores (D8281) |
| Polyethelineimine (PEI) | Sigma (P3143) |
| 96 well Unifilter plate GF/C | Packard (6005174) |
| Topseal A | Packard (6005185) |
| Microscint-O | Packard (6013611) |
| SR49059 (UK222,633) | Compound Control |

Equipment:
Packard Unifilter Unit
Top Counter/NXT Counter

ii) Methods

Working Solutions

| | |
|---|---|
| Membrane Preparation Buffer: | 25 mM HEPES (pH 7.4 |
| | 5 mM MgCl₂ |
| | Protease inhibitors (1 tablet per 50 ml) |
| Freezing Buffer: | 25 mM HEPES (pH 7.4) |
| | 5 mM MgCl₂ |
| | 20% Glycerol |
| Assay Buffer: | 25 mM HEPES (pH 7.4) |
| | 5 mM MgCl₂ |
| | 0.05% BSA |
| Wash Buffer | 25 mM HEPES (pH 7.4) |
| | 5 mM MgCl₂ |
| ³H-AVP | 5 nM solution in assay buffer (for final assay concentration of |
| | 0.5 nM). |
| Totals | 25% DMSO in ddH₂O, |
| NSB | 10 μM βMCPVP in 25% DMSO/ddH₂O (for final assay |
| | concentration of 1 μM). |
| STD | SR49059 (UK222,633) will be diluted in 25% DMSO/ddH₂O |
| | starting with a top concentration of 1 μM (for final assay |
| | concentration of 100 nM) and continuing in 0.5 Log steps down to |
| | 30 pM (for final assay concentration of 3 pM). |
| Compounds: | 50 μl of compound at 4 mM in 100% DMSO. This will be diluted 4 |
| | fold in dH₂O to give a top concentration of 1 mM in 25% |
| | DMSO. Compounds will be further diluted in half log steps in 25% |
| | DMSO except the first dilution (1 mM to 300 μM) which will be |
| | in 18% DMSO (25% DMSO after dilution). Dilutions will be |
| | performed by hand or using the Tecan and protocol file |
| | Kin28IC50dilution2.gem. 10 pt IC50 curves will be started at lower |
| | concentrations as required by compound but all drugs will be |
| | screened initially starting at 100 μM. |
| 0.5% PEI | 50% PEI prepared in distilled H₂O, diluted to 0.5% in dH₂O |

iii) Membrane Preparation
Frozen cell pellets are retrieved from the freezer and thawed gently on ice.
3 ml of membrane prep buffer per ml of original packed cell volume is
added and the suspension homogenised with a polytron for several bursts
of 5 seconds on ice until well dispersed before centrifuging at 1000 rpm for 10 mins
The supernatant is removed and stored on ice. A further 3 ml of membrane prep buffer per ml of original packed cell volume is added to the pellet, homogenised on ice and then centrifuged at 1000 rpm for 10 mins
The supernatant is removed, added to the previously removed volume of supernatant and then centrifuged at 25,000×g and 4° C. for 30 mins
The 25,000×g pellet is re-suspended by homogenisation in 1 ml of freezing buffer per ml of original packed cell volume and the protein concentration determined

iv) Determination Of Protein Concentration
BSA is prepared in distilled H₂O at the following concentrations: 2000, 1000, 500, 250, 125, 62.5 and 31.25 5 μg/ml
10 μl of the each of the BSA solutions are added to a clear 96 well plate in triplicate (see plate map in appendix), and 10 μl of distilled H₂O is added to three blank wells
10 μl of the membrane prep is added to the plate in triplicate as are 1-in-3,1-in-10, 1-in-30 and 1-in-100 dilutions of the membrane prep
200 μl of Pierce Protein reagent (50 A:1 B) is added to each well and the plate is incubated 30 minutes at 37° C. then read on a Anthos spectrophotometer at an absorbance setting of 570 nm
From the BSA standard curve, the concentration of protein in the membrane prep is determined (the dilution of the membrane prep which lies on the centre of the standard curve is used).
The membrane prep is diluted to a protein concentration of 5 mg/ml in freezing buffer before being frozen in 200 μl aliquots at −80° C.

v) Assay Protocol
Assay reagents are prepared (³H-AVP, βMCPVP (NSB compound) and test compounds—see working solutions above). Any peptide solutions are kept on ice.
The plate format will be 10 point IC50-4 compounds per plate duplicate separate rows. The following reagents are added to the appropriate wells of a 96 well polypropylene block and vortexed

| | |
|---|---|
| To each Total well (T): | 25 μl ³H-AVP |
| (A1, B1, C1, D12E12, F12, G12 & H12) | 25 μl vehicle |
| To each NSB well (N): | 25 μl ³H-AVP |
| (A12, B12, C12, D12, E1, F1, G1 & H1) | 25 μl βMCPVP |
| To each assay well: | 25 μl ³H-AVP |
| | 25 μl test compound |

| | 1 | 2 | 3 | 4 | 5 | 6 | 7 | 8 | 9 | 10 | 11 | 12 |
|---|---|---|---|---|---|---|---|---|---|---|---|---|
| A | T | C1 | C2 | C3 | C4 | C5 | C6 | C7 | C8 | C9 | C10 | N |
| B | T | C1 | C2 | C3 | C4 | C5 | C6 | C7 | C8 | C9 | C10 | N |
| C | T | C1 | C2 | C3 | C4 | C5 | C6 | C7 | C8 | C9 | C10 | N |
| D | T | C1 | C2 | C3 | C4 | C5 | C6 | C7 | C8 | C9 | C10 | N |
| E | N | C1 | C2 | C3 | C4 | C5 | C6 | C7 | C8 | C9 | C10 | T |
| F | N | C1 | C2 | C3 | C4 | C5 | C6 | C7 | C8 | C9 | C10 | T |
| G | N | C1 | C2 | C3 | C4 | C5 | C6 | C7 | C8 | C9 | C10 | T |
| H | N | C1 | C2 | C3 | C4 | C5 | C6 | C7 | C8 | C9 | C10 | T |

C1 = Concentration 1,
C2 = Concentration 2,
C3 = Concentration 3, etc.
Row A&B = STD
Row C&D = compound 1
Row E&F = compound 2
Row G&H = compound 3

Membrane protein is thawed gently on ice and diluted to the optimum protein concentration for the assay (see appendix for protein linearity determination) (approximately 100 μg/ml)
200 μl of membrane protein is added to each well to initiate the reaction and the blocks are then incubated shaking gently at RT for 60 mins
The reaction is terminated by filtration through Unifilter GF/C filters pre-soaked in 0.5% PEI and rapid washing with 3×1 ml ice cold wash buffer
The filters are dried for 2 hours in a 55° C. oven or left overnight (−16 hours) on the bench
The filters are sealed on the bottom and 30 μl of Microscint-O is placed in each well. The filters are then sealed with Topseal A and counted on a Packard TopCounter (Bid 503/G7A) using a [³H] 96 well Unifiter protocol 11.

### C. Data Analysis

Data Analysis Carried Out by ECADA

Specific binding is calculated as follows:

Specific binding=mean Total cpm−mean NSB cpm

For the test compounds, the amount of ligand bound to the receptor is expressed as follows:

% bound=(sample cpm−mean NSB cpm)/specific binding cpm×100

The percentage inhibition of ligand binding is reported with the % inhibition being is calculated as follows:

% inhibition=100−% bound

### Oxytocin Antagonist Functional Assay and Vasopressin V1a Antagonist Functional Assay

Spontaneous contractions of myometrium from humans, non-human primates and rodents are sensitive to selective oxytocin receptor antagonism in vitro (see Wilson et al BJOG 2001 September; 108(9):960-6).

In vitro pharmacology of spontaneous contractions of myometrium from humans and animals. Samples of human myometrium were obtained at caesarian section. Tissue strips were suspended in organ baths for isometric force recording. Cumulative concentration effect curves to a selective oxytocin receptor antagonist and a mixed oxytocin/vasopressin V1a receptor antagonist may be obtained. The inhibition of spontaneous myometrial contractions in vitro is observed.

### Combinations

In more detail, the present invention further comprises the combination of a compound of the invention for the treatment of a male ejaculatory disorder, in particular premature ejaculation, as outlined herein with one or more of auxiliary active agents (see later discussion for suitable examples).

The present invention further comprises the use of a combination consisting essentially of a selective oxytocin antagonist according to the present invention and two auxiliary active agents (see later discussion for suitable examples) in the manufacture of a medicament for the treatment and/or prevention of a male ejaculatory disorder, in particular premature ejaculation, as outlined herein.

The present invention further comprises the use of a combination consisting of a selective oxytocin antagonist according to the present invention and two auxiliary active agents (see later discussion for suitable examples) in the manufacture of a medicament for the treatment and/or prevention of a male ejaculatory disorder, in particular premature ejaculation, as outlined herein.

The present invention further comprises the use of a combination consisting essentially of a selective oxytocin antagonist according to the present invention and one auxiliary active agent (see later discussion for suitable examples) in the manufacture or preparation of a medicament for the treatment and/or prevention of a male ejaculatory disorder, in particular premature ejaculation, as outlined herein.

The present invention further comprises the use of a combination consisting of a selective oxytocin antagonist according to the present invention and one auxiliary active agent (see later discussion for suitable examples) in the manufacture or preparation of a medicament for the treatment and/or prevention of a male ejaculatory disorder, in particular premature ejaculation, as outlined herein.

Thus a further combination aspect of the invention provides a pharmaceutical combination (for simultaneous, separate or sequential administration) comprising a compound of the invention and one or more auxiliary active agents (see later discussion for suitable examples).

A yet further combination aspect of the invention provides a pharmaceutical composition (for simultaneous, separate or sequential administration) consisting essentially of a selective oxytocin antagonist and two auxiliary active agents (see later discussion for suitable examples).

A yet further combination aspect of the invention provides a pharmaceutical composition (for simultaneous, separate or sequential administration) consisting of a selective oxytocin antagonist and two auxiliary active agents (see later discussion for suitable examples).

A yet further combination aspect of the invention provides a pharmaceutical composition (for simultaneous, separate or sequential administration) consisting essentially of a selective oxytocin antagonist and one auxiliary active agent (see later discussion for suitable examples).

A yet further combination aspect of the invention provides a pharmaceutical composition (for simultaneous, separate or sequential administration) consisting of a selective oxytocin antagonist and one auxiliary active agent (see later discussion for suitable examples).

### Auxiliary Active Agents

Suitable auxiliary active agents for use in the combinations of the present invention include:
1) A PDE inhibitor, more particularly a PDE 5 inhibitor (see hereinafter), said inhibitors preferably having an IC50 against the respective enzyme of less than 100 nM;
2) A serotonin receptor agonist or modulator, more particularly agonists or modulators for 5HT2C, 5HT1 B and/or 5HT1 D receptors, including anpirtoline;
3) A serotonin receptor antagonist or modulator, more particularly antagonists or modulators for 5HT1A, including NAD-299 (robalzotan) and WAY-100635, and/or more particularly antagonists or modulators for 5HT3 receptors, including batanopirde, granisetron, ondansetron, tropistron and MDL-73147EF;
4) An antidepressant, in particular i) a selective serotonin re-uptake inhibitor (SSRI), including sertraline, fluoxetine, fluvoxamine, paroxetine, citalopram, venlafaxine, mirtazapine, nefazodone and trazodone; ii) a tricyclic antidepressant (TCA), including clomipramine, desapramine, imipramine, amitriptyline, doxepine, amoxapine, maprotiline, nortriptyline, protriptyline, trimipramine and buproprion; and iii) monoamine oxidase;
5) An α-adrenergic receptor antagonist (also known as α-adrenergic blockers, α-blockers or α-receptor blockers); suitable α1-adrenergic receptor antagonists include: phentolamine, prazosin, phentolamine mesylate, trazodone, alfuzosin, indoramin, naftopidil, tamsulosin, phenoxybenzamine, rauwolfa alkaloids, Recordati 15/2739, SNAP 1069, SNAP 5089, RS17053, SL 89.0591, doxazosin, terazosin and abanoquil; suitable α2-adrenergic receptor antagonists include dibenamine, tolazoline, trimazosin, efaroxan, yohimbine, idazoxan clonidine and dibenamine; suitable non-selective α-adrenergic receptor antagonists include dapiprazole; further α-adrenergic receptor antagonists are described in WO99/30697, U.S. Pat. No. 4,188,390, U.S. Pat. No. 4,026,894, U.S. Pat. No. 3,511,836, U.S. Pat. No. 4,315,007, U.S. Pat. No. 3,527,761, U.S. Pat. No. 3,997,666, U.S. Pat. No. 2,503,059, U.S. Pat. No. 4,703,063, U.S. Pat. No. 3,381,009, U.S. Pat. No. 4,252,721 and U.S. Pat. No. 2,599,000 each of which is incorporated herein by reference;
6) A rapid onset selective serotonin re-uptake inhibitor (rapid onset SSRI), such as 3-[(Dimethylamino)methyl]-4-[4-(methylsulfanyl)phenoxy]benzenesulfonamide (as published in WO01/72687—Example 28), for example.

By cross reference herein to compounds contained in patents and patent applications which can be used in accordance with invention, we mean the therapeutically active compounds as defined in the claims (in particular of claim 1) and the specific examples (all of which is incorporated herein by reference).

If a combination of active agents is administered, then they may be administered simultaneously, separately or sequentially.

### Auxiliary Agents—PDE5 Inhibitors

Suitable cGMP PDE5 inhibitors for the use according to the present invention include:

the pyrazolo[4,3-d]pyrimidin-7-ones disclosed in EP-A-0463756; the pyrazolo[4,3-d]pyrimidin-7-ones disclosed in EP-A-0526004; the pyrazolo[4,3-d]pyrimidin-7-ones disclosed in published international patent application WO 93/06104; the isomeric pyrazolo[3,4-d]pyrimidin-4-ones disclosed in published international patent application WO 93/07149; the quinazolin-4-ones disclosed in published international patent application WO 93/12095; the pyrido[3,2-d]pyrimidin-4-ones disclosed in published international patent application WO 94/05661; the purin-6-ones disclosed in published international patent application WO 94/00453; the pyrazolo[4,3-d]pyrimidin-7-ones disclosed in published international patent application WO 98/49166; the pyrazolo[4,3-d]pyrimidin-7-ones disclosed in published international patent application WO 99/54333; the pyrazolo[4,3-d]pyrimidin-4-ones disclosed in EP-A-0995751; the pyrazolo[4,3-d]pyrimidin-7-ones disclosed in published international patent application WO 00/24745; the pyrazolo[4,3-d]pyrimidin-4-ones disclosed in EP-A-0995750; the compounds disclosed in published international application WO95/19978; the compounds disclosed in published international application WO 99/24433 and the compounds disclosed in published international application WO 93/07124.

The pyrazolo[4,3-d]pyrimidin-7-ones disclosed, in published international application WO 01/27112; the pyrazolo[4,3-d]pyrimidin-7-ones disclosed in published international application WO 01/27113; the compounds disclosed in EP-A-1092718 and the compounds disclosed in EP-A-1092719.

Preferred type V phosphodiesterase inhibitors (=phosphodiesterase 5 (PDE) inhibitors; PDE5i's) for the use according to the present invention include:

5-[2-ethoxy-5-(4-methyl-1-piperazinylsulphonyl)phenyl]-1-methyl-3-n-propy-I-1,6-dihydro-7H-pyrazolo[4,3-d]pyrimidin-7-one (sildenafil) also known as 1-[[3-(6,7-dihydro-1-methyl-7-oxo-3-propyl-1H-pyrazolo[4,3-d]pyrimidin-5-yl)-4-ethoxyphenyl]sulphonyl]-4-methylpiperazine (see EP-A-0463756);

5-(2-ethoxy-5-morpholinoacetylphenyl)-1-methyl-3-n-propyl-1,6-dihydro-7H-pyrazolo[4,3-d]pyrimidin-7-one (see EP-A-0526004);

3-ethyl-5-[5-(4-ethylpiperazin-1-ylsulphonyl)-2-n-propoxyphenyl]-2-(pyridin-2-yl)methyl-2,6-dihydro-7H-pyrazolo[4,3-d]pyrimidin-7-one (see WO98/49166);

3-ethyl-5-[5-(4-ethylpiperazin-1-ylsulphonyl)-2-(2-methoxyethoxy)pyridin-3-yl]-2-(pyridin-2-yl)methyl-2,6-dihydro-7H-pyrazolo[4,3-d]pyrimidin-7-one (see WO99/54333);

(+)-3-ethyl-5-[5-(4-ethyl piperazin-1-ylsulphonyl)-2-(2-methoxy-1(R)-methylethoxy)pyridin-3-yl]-2-methyl-2,6-dihydro-7H-pyrazolo[4,3-d]pyrimidin-7-one, also known as 3-ethyl-5-{5-[4-ethylpiperazin-1-ylsulphonyl]-2-([(1R)-2-methoxy-1-methyl-ethyl]oxy)pyridin-3-yl}-2-methyl-2,6-dihydro-7H-pyrazolo[4,3-d]pyrimidin-7-one (see WO99/54333);

5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, also known as 1-{6-ethoxy-5-[3-ethyl-6,7-dihydro-2-(2-methoxyethyl)-7-oxo-2H-pyrazolo[4-,3-d]pyrimidin-5-yl]-3-pyridylsulphonyl}-4-ethylpiperazine (see WO 01/27113, Example 8);

5-[2-iso-Butoxy-5-(4-ethylpiperazin-1-ylsulphonyl)pyridin-3-yl]-3-ethyl-2-(1-methylpiperidin-4-yl)-2,6-dihydro-7H-pyrazolo[4,3-d]pyrimidin-7-one (see WO 01/27113, Example 15);

5-[2-Ethoxy-5-(4-ethylpiperazin-1-ylsulphonyl)pyridin-3-yl]-3-ethyl-2-phenyl-2,6-dihydro-7H-pyrazolo[4,3-d]pyrimidin-7-one (see WO 01/27113, Example 66);

5-(5-Acetyl-2-propoxy-3-pyridinyl)-3-ethyl-2-(1-isopropyl-3-azetidinyl)-2-,6-dihydro-7H-pyrazolo[4,3-d]pyrimidin-7-one (see WO 01/27112, Example 124);

5-(5-Acetyl-2-butoxy-3-pyridinyl)-3-ethyl-2-(1-ethyl-3-azetidinyl)-2,6-dihydro-7H-pyrazolo[4,3-d]pyrimidin-7-one (see WO 01/27112, Example 132);

(6R,12aR)-2,3,6,7,12,12a-hexahydro-2-methyl-6-(3,4-methylenedioxyphenyl)-pyrazino[2′,1′:6,1]pyrido[3,4-b]indole-1,4-dione (IC-351), i.e. the compound of examples 78 and 95 of published international application WO95/19978, as well as the compound of examples 1, 3, 7 and 8;

2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil) also known as 1-[[3-(3,4-dihydro-5-methyl-4-oxo-7-propylimidazo[5,1-f]-as-triazin-2-yl)-4-ethoxyphenyl]sulphonyl]-4-ethylpiperazine, i.e. the compound of examples 20, 19, 337 and 336 of published international application WO99/24433; and the compound of example 11 of published international application WO93/07124 (EISAI);

and compounds 3 and 14 from Rotella D P, J. Med. Chem., 2000, 43, 1257.

Still other type cGMP PDE5 inhibitors useful in conjunction with the present invention include: 4-bromo-5-(pyridylmethylamino)-6-[3-(4-chlorophenyl)-propoxy]-3(2H)pyridazinone; 1-[4-[(1,3-benzodioxol-5-ylmethyl)amino]-6-chloro-2-quinozolinyl]-4-piperidine-carboxylic acid, monosodium salt; (+)-cis-5,6a,7,9,9,9a-hexahydro-2-[4-(trifluoromethyl)-phenylmethyl-5-methyl-cyclopent-4,5]imidazo[2,1-b]purin-4(3H)one; furazlocillin; cis-2-hexyl-5-methyl-3,4,5,6a,7,8,9,9a-octahydrocyclopent[4,5]-imidazo[2,-1-b]purin-4-one; 3-acetyl-1-(2-chlorobenzyl)-2-propylindole-6-carboxylate; 3-acetyl-1-(2-chlorobenzyl)-2-propylindole-6-carboxylate; 4-bromo-5-(3-pyridyl methylamino)-6-(3-(4-chlorophenyl)propoxy)-3-(2H)pyridazinone; 1-methyl-5(5-morpholinoacetyl-2-n-N propoxyphenyl)-3-n-propyl-1,6-dihydro-7H-pyrazolo(4,3-d)pyrimidin-7-one; 1-[4-[(1,3-benzodioxol-5-ylmethyl)amino]-6-chloro-2-quinazolinyl]-4-piper-idinecarboxylic acid, monosodium salt; Pharmaprojects No. 4516 (Glaxo Wellcome); Pharmaprojects No. 5051 (Bayer); Pharmaprojects No. 5064 (Kyowa Hakko; see WO 96/26940); Pharmaprojects No. 5069 (Schering Plough); GF-196960 (Glaxo Wellcome); E-8010 and E-4010 (Eisai); Bay-38-3045 & 38-9456 (Bayer) and Sch-51866.

The suitability of any particular cGMP PDE5 inhibitor can be readily determined by evaluation of its potency and selectivity using literature methods followed by evaluation of its toxicity, absorption, metabolism, pharmacokinetics, etc in accordance with standard pharmaceutical practice.

Preferably, the cGMP PDE5 inhibitors have an IC₅₀ at less than 100 nanomolar, more preferably, at less than 50 nanomolar, more preferably still at less than 10 nanomolar.

IC50 values for the cGMP PDE5 inhibitors may be determined using the PDE5 assay in the Test Methods Section hereinafter.

Preferably the cGMP PDE5 inhibitors used in the pharmaceutical combinations according to the present invention are selective for the PDE5 enzyme. Preferably they have a selectivity of PDE5 over PDE3 of greater than 100 more preferably greater than 300. More preferably the PDE5 has a selectivity over both PDE3 and PDE4 of greater than 100, more preferably greater than 300.

Selectivity ratios may readily be determined by the skilled person.

It is to be understood that the contents of the above published patent applications, and in particular the general formulae and exemplified compounds therein are incorporated herein in their entirety by reference thereto.

### Corpus Cavernosum

As used herein, the term “corpus cavernosum” refers inter alia to a mass of tissue found in the penis. In this regard, the body of the penis is composed of three cylindrical masses of tissue, each surrounded by fibrous tissue called the tunica albuginea. The paired dorsolateral masses are called the corpora cavernosa penis (corpora=main bodies; cavernosa=hollow); the smaller midventral mass, the corpus spongiosum penis contains the spongy urethra and functions in keeping the spongy urethra open during ejaculation. All three masses are enclosed by fascia and skin and consist of erectile tissue permeated by blood sinuses. The corpus cavernosum comprises smooth muscle cells.

### Ejaculation

Ejaculation comprises two separate components—emission and ejaculation. Emission is the deposition of seminal fluid and sperm from the distal epididymis, vas deferens, seminal vesicles and prostrate into the prostatic urethra.

Subsequent to this deposition is the forcible expulsion of the seminal contents from the urethral meatus. Ejaculation is distinct from orgasm, which is purely a cerebral event. Often the two processes are coincidental.

### Penile Erection

As used herein, the term “penile erection” refers to the situation whereby, upon stimulation, which may be visual, tactile, auditory, olfactory or from the imagination, the arteries supplying the penis dilate and large quantities of blood enter the blood sinuses. Expansion of these spaces compresses the veins draining the penis, so blood outflow is slowed. These vascular changes, due to a parasympathetic reflex, result in an erection. The penis returns to its flaccid state when the arteries constrict and pressure on the veins is relieved.

### Smooth Muscle

As used herein, the term “smooth muscle” refers to a tissue specialised for contraction composed of smooth muscle fibres (cells) which are located in the walls of hollow internal organs and innervated by autonomic motor neurons. The term “smooth muscle” means muscle lacking striations, hence giving it a smooth appearance. It is also called involuntary muscle. An increase in the concentration of Ca²⁺ in smooth muscle cytosol initiates contraction, just as in striated muscle. However, sacroplasmic reticulum (the reservoir for Ca²⁺ in striated muscle) is scanty in smooth muscle. Calcium ions flow into smooth muscle cytosol from both the extracellular fluid and sarcoplasmic reticulum, but because there are no transverse tubules in smooth muscle fibres, it takes longer for Ca²⁺ to reach the filaments in the centre of the fibre and trigger the contractile process. This accounts, in part, for the slow onset and prolonged contraction of smooth muscle.

### Contraction and Relaxation

Several mechanisms regulate contraction and relaxation of smooth muscle cells. In one, a regulatory protein called calmodulin binds to Ca²⁺ in the cytosol. Not only do calcium ions enter smooth muscle fibres slowly, but they also move slowly out of the muscle fibre when excitation declines, which delays relaxation. The prolonged presence of Ca²⁺ in the cytosol provides for smooth muscle tone, a state of continued partial contraction. Smooth muscle tissue is located in the walls of hollow internal organs such as blood vessels, airways to the lungs, the stomach, intestinal gall bladder, urinary bladder, the corpus cavernosa of the penis and the clitoris.

### Treatment

It is to be appreciated that all references herein to treatment include one or more of curative, palliative and prophylactic treatment.

### Sexual Stimulation

The present invention also encompasses use as defined hereinbefore via administration of a selective oxytocin antagonist (and an auxiliary agent where applicable) before and/or during sexual stimulation. Here the term “sexual stimulation” may be synonymous with the term “sexual arousal”. This aspect of the present invention is advantageous because it provides systemic (physiological) selectivity.

Thus, according to the present invention it is highly desirable that there is a sexual stimulation step at some stage. Here, “sexual stimulation” may be one or more of a visual stimulation, a physical stimulation, an auditory stimulation, or a thought stimulation.

### Agent

Agents for use in the treatment of a male ejaculatory disorder, in particular premature ejaculation, according to the present invention may be any suitable agent that can act as a selective oxytocin antagonist and, where appropriate a combination of a selective oxytocin antagonist and an auxiliary agent as detailed hereinbefore. As used herein, the term “agent” includes any entity capable of selectively inhibiting oxytocin receptors.

Such agents (i.e. the agents as defined above) can be an organic compound or other chemical. The substance may even be an amino acid sequence or a chemical derivative thereof. The agent may even be a nucleotide sequence—which may be a sense sequence or an anti-sense sequence. The agent may even be an antibody.

Thus, the term “agent” includes, but is not limited to, a compound which may be obtainable from or produced by any suitable source, whether natural or not.

The agent may be designed or obtained from a library of compounds which may comprise peptides, as well as other compounds, such as small organic molecules, such as lead compounds.

By way of example, the agent may be a natural substance, a biological macromolecule, or an extract made from biological materials such as bacteria, fungi, or animal (particularly mammalian) cells or tissues, an organic or an inorganic molecule, a synthetic agent, a semi-synthetic agent, a structural or functional mimetic, a peptide, a peptidomimetics, a derivatised agent, a peptide cleaved from a whole protein, or a peptide synthesised synthetically (such as, by way of example, either using a peptide synthesiser or by recombinant techniques or combinations thereof, a recombinant agent, an antibody, a natural or a non-natural agent, a fusion protein or equivalent thereof and mutants, derivatives or combinations thereof.

As used herein, the term “agent” may be a single entity or it may be a combination of agents.

If the agent is an organic compound then for some applications that organic compound may typically comprise two or more linked hydrocarbyl groups. For some applications, preferably the agent comprises at least two cyclic groups—optionally wherein one of which cyclic groups may be a fused cyclic ring structure. For some applications, at least one of the cyclic groups is a heterocyclic group. For some applications, preferably the heterocyclic group comprises at least one N in the ring. An example of such a compound is presented herein.

The agent may contain one or more of alkyl, alkoxy, alkenyl, alkylene and alkenylene groups—which may be unbranched- or branched-chain.

### Substituted

For the avoidance of doubt, unless otherwise indicated, the term substituted means substituted by one or more defined groups. In the case where groups may be selected from a number of alternative groups, the selected groups may be the same or different. For the avoidance of doubt, the term independently means that where more than one substituent is selected from a number of possible substituents, those substituents may be the same or different.

### Pharmaceutically Acceptable Salt

The agent may be in the form of and/or may be administered as-a pharmaceutically acceptable salt-such as an acid addition salt or a base salt-or a solvate thereof, including a hydrate thereof. For a review on suitable salts see Berge et al., J. Pharm. Sci., 1977, 66, 1-19.

Typically, a pharmaceutically acceptable salt may be readily prepared by using a desired acid or base, as appropriate. The salt may precipitate from solution and be collected by filtration or may be recovered by evaporation of the solvent.

Suitable acid addition salts are formed from acids which form non-toxic salts and examples are the hydrochloride, hydrobromide, hydroiodide, sulphate, bisulphate, nitrate, phosphate, hydrogen phosphate, acetate, maleate, fumarate, lactate, tartrate, citrate, gluconate, succinate, saccharate, benzoate, methanesulphonate, ethanesulphonate, benzenesulphonate, p-toluenesulphonate and pamoate salts.

Suitable base salts are formed from bases which form non-toxic salts and examples are the sodium, potassium, aluminium, calcium, magnesium, zinc and diethanolamine salts.

### Polymorphic Forms(s)/Asymmetric Carbon(s)

The agent may exist in polymorphic form.

The agent may contain one or more asymmetric carbon atoms and therefore exists in two or more stereoisomeric forms. Where an agent contains an alkenyl or alkenylene group, cis (E) and trans (Z) isomerism may also occur. The present invention includes the individual stereoisomers of the agent and, where appropriate, the individual tautomeric forms thereof, together with mixtures thereof.

Separation of diastereoisomers or cis and trans isomers may be achieved by conventional techniques, e.g. by fractional crystallisation, chromatography or H.P.L.C. of a stereoisomeric mixture of the agent or a suitable salt or derivative thereof. An individual enantiomer of the agent may also be prepared from a corresponding optically pure intermediate or by resolution, such as by H.P.L.C. of the corresponding racemate using a suitable chiral support or by fractional crystallisation of the diastereoisomeric salts formed by reaction of the corresponding racemate with a suitable optically active acid or base, as appropriate.

### Isptopic Variations

The present invention also includes all suitable isotopic variations of the agent or a pharmaceutically acceptable salt thereof. An isotopic variation of an agent of the present invention or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations of the agent and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the agent and pharmaceutically acceptable salts thereof can generally be prepared by conventional procedures using appropriate isotopic variations of suitable reagents.

### Produgs

It will be appreciated by those skilled in the art that the agent may be derived from a prodrug. Examples of prodrugs include entities that have certain protected group(s) and which may not possess pharmacological activity as such, but may, in certain instances, be administered (such as orally or parenterally) and thereafter metabolised in the body to form the agent which are pharmacologically active.

### Pro-Moieties

It will be further appreciated that certain moieties known as “pro-moieties”, for example as described in “Design of Prodrugs” by H. Bundgaard, Elsevier, 1985 (the disclosure of which is hereby incorporated by reference), may be placed on appropriate functionalities of the agents. Such prodrugs are also included within the scope of the invention.

### Inhibitor/Antagonist

The term antagonist as used herein in relation to the selective oxytocin antagonist is to be regarded as being interchangeable with the term inhibitor. Likewise, the term inhibitor as used herein, in relation to the auxiliary agents hereinbefore presented for example (such as where applicable PDEi or PDE5i compounds), is to be regarded as being interchangeable with the term antagonist.

As used herein, the term “antagonist” means any agent that reduces the action of another agent or target. The antagonistic action may result from a combination of the substance being antagonised (chemical antagonism) or the production of an opposite effect through a different target (functional antagonism or physiological antagonism) or as a consequence of competition for the binding site of an intermediate that links target activation to the effect observed (indirect antagonism).

### Pharmaceutical Compositions

The present invention also provides a pharmaceutical composition comprising a therapeutically effective amount of the agent of the present invention and a pharmaceutically acceptable carrier, diluent or excipient (including combinations thereof).

The pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilisers, dyes and even flavouring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition of the present invention may be formulated to be delivered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be delivered by both routes.

Where the agent is to be delivered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

For some embodiments, the agents of the present invention may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in WO-A-91/11172, WO-A-94/02518 and WO-A-98/55148.

In a preferred embodiment, the agents of the present invention are delivered systemically (such as orally, buccally, sublingually), more preferably orally.

Hence, preferably the agent is in a form that is suitable for oral delivery.

### Administration

The term “administered” includes delivery by viral or non-viral techniques. Viral delivery mechanisms include but are not limited to adenoviral vectors, adeno-associated viral (AAV) vectors, herpes viral vectors, retroviral vectors, lentiviral vectors, and baculoviral vectors. Non-viral delivery mechanisms include lipid mediated transfection, liposomes, immunoliposomes, lipofectin, cationic facial amphiphiles (CFAs) and combinations thereof.

The agents of the present invention may be administered alone but will generally be administered as a pharmaceutical composition-e.g. when the agent is in admixture with a suitable pharmaceutical excipient, diluent or carrier selected with regard to the intended route of administration and standard pharmaceutical practice.

For example, the agent can be administered (e.g. orally or topically) in the form of tablets, capsules, ovules, elixirs, solutions or suspensions, which may contain flavouring or colouring agents, for immediate-, delayed-, modified-, sustained-, pulsed- or controlled-release applications.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavouring agents, colouring matter or dyes, with emulsifying and/or suspending agents and with diluents such as water, ethanol, propylene glycol and glycerin, and combinations thereof.

The routes for administration (delivery) include, but are not limited to, one or more of: oral (e.g. as a tablet, capsule, or as an ingestable solution), topical, mucosal (e.g. as a nasal spray or aerosol for inhalation), nasal, parenteral (e.g. by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, penile, vaginal, epidural, sublingual.

It is to be understood that not all of the agents need be administered by the same route. Likewise, if the composition comprises more than one active component, then those components may be administered by different routes.

If the agent of the present invention is administered parenterally, then examples of such administration include one or more of: intravenously, intra-arterially, intraperitoneally, intrathecally, intraventricularly, intraurethrally, intrasternally, intracranially, intramuscularly or subcutaneously administering the agent; and/or by using infusion techniques.

For parenteral administration, the agent is best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

As indicated, the agent of the present invention can be administered intranasally or by inhalation and is conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoroethane, a hydrofluoroalkane such as 1,1,1,2-tetrafluoroethane (HFA134A™) or 1,1,1,2,3,3,3-heptafluoropropane (HFA 227EA™), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of the agent and a suitable powder base such as lactose or starch.

Alternatively, the agent of the present invention can be administered in the form of a suppository or pessary, or it may be applied topically in the form of a gel, hydrogel, lotion, solution, cream, ointment or dusting powder. The agent of the present invention may also be dermally or transdermally administered, for example, by the use of a skin patch. They may also be administered by the pulmonary or rectal routes. They may also be administered by the ocular route. For ophthalmic use, the compounds can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the agent of the present invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, it can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

The compositions of the present invention may be administered by direct injection.

For some applications, preferably the agent is administered orally.

For some applications, preferably the agent is administered topically.

### Dose Levels

Typically, a physician will determine the actual dosage which will be most suitable for an individual subject. The specific dose level and frequency of dosage for any particular individual may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The agent and/or the pharmaceutical composition of the present invention may be administered in accordance with a regimen of from 1 to 10 times per day, such as once or twice per day.

For oral and parenteral administration to humans, the daily dosage level of the agent may be in single or divided doses.

Depending upon the need, the agent may be administered at a dose of from 0.01 to 30 mg/kg body weight, such as from 0.1 to 10 mg/kg, more preferably from 0.1 to 1 mg/kg body weight. Naturally, the dosages mentioned herein are exemplary of the average case. There can, of course, be individual instances where higher or lower dosage ranges are merited.

Typically the daily oral dose may be, for instance, between 20-1000 mg, preferably 50-300 mg for example.

### Formulation

The agents of the present invention may be formulated into a pharmaceutical composition, such as by mixing with one or more of a suitable carrier, diluent or excipient, by using techniques that are known in the art.

The following present some non-limiting examples of formulations.

Formulation 1: A tablet is prepared using the following ingredients:

| | weight/mg |
|---|---|
| | g |
| Agent | 250 |
| Cellulose, microcrystalline | 400 |
| Silicon dioxide, fumed | 10 |
| Stearic acid | 5 |
| Total | 665 |

the components are blended and compressed to form tablets each weighing 665 mg.

Formulation 2: An intravenous formulation may be prepared as follows:

| | |
|---|---|
| Agent | 100 mg |
| Isotonic saline | 1,000 ml |

### Individual

As used herein, the term “individual” refers to vertebrates, particularly members of the mammalian species. The term includes but is not limited to domestic animals, sports animals, primates and humans.

### Bioavailability

Preferably, the compounds of the invention (and combinations) are orally bioavailable. Oral bioavailability refers to the proportion of an orally administered drug that reaches the systemic circulation. The factors that determine oral bioavailability of a drug are dissolution, membrane permeability and metabolic stability. Typically, a screening cascade of firstly in vitro and then in vivo techniques is used to determine oral bioavailability.

Dissolution, the solubilisation of the drug by the aqueous contents of the gastrointestinal tract (GIT), can be predicted from in vitro solubility experiments conducted at appropriate pH to mimic the GIT. Preferably the compounds of the invention have a minimum solubility of 50 mg/ml. Solubility can be determined by standard procedures known in the art such as described in Adv. Drug Deliv. Rev. 23, 3-25, 1997.

Membrane permeability refers to the passage of the compound through the cells of the GIT. Lipophilicity is a key property in predicting this and is defined by in vitro Log D_{7.4} measurements using organic solvents and buffer. Preferably the compounds of the invention have a Log D_{7.4} of −2 to +4, more preferably −1 to +2. The log D can be determined by standard procedures known in the art such as described in J. Pharm. Pharmacol. 1990, 42:144.

Cell monolayer assays such as CaCO₂ add substantially to prediction of favourable membrane permeability in the presence of efflux transporters such as p-glycoprotein, so-called caco-2 flux. Preferably, compounds of the invention have a caco-2 flux of greater than 2×10⁻⁶ cms⁻¹, more preferably greater than 5×10⁻⁶ cms⁻¹. The caco flux value can be determined by standard procedures known in the art such as described in J. Pharm. Sci., 1990, 79, 595-600.

Metabolic stability addresses the ability of the GIT or the liver to metabolise compounds during the absorption process: the first pass effect. Assay systems such as microsomes, hepatocytes etc are predictive of metabolic liability. Preferably the compounds of the Examples show metabolic stability in the assay system that is commensurate with an hepatic extraction of less then 0.5. Examples of assay systems and data manipulation are described in Curr. Opin. Drug Disc. Devel., 201, 4, 36-44, Drug Met. Disp., 2000, 28, 1518-1523.

Because of the interplay of the above processes further support that a drug will be orally bioavailable in humans can be gained by in vivo experiments in animals. Absolute bioavailability is determined in these studies by administering the compound separately or in mixtures by the oral route. For absolute determinations (% absorbed) the intravenous route is also employed. Examples of the assessment of oral bioavailability in animals can be found in Drug Met. Disp., 2001, 29, 82-87; J. Med Chem, 1997, 40, 827-829, Drug Met. Disp., 1999, 27, 221-226.

### Chemical Synthesis Methods

Typically the selective oxytocin antagonist (and/or PDEi/PDE5i where applicable) suitable for the use according to the present invention will be prepared by chemical synthesis techniques.

The agent or target or variants, homologues, derivatives, fragments or mimetics thereof may be produced using chemical methods to synthesise the agent in whole or in part. For example, peptides can be synthesised by solid phase techniques, cleaved from the resin, and purified by preparative high performance liquid chromatography (e.g., Creighton (1983) Proteins Structures And Molecular Principles, WH Freeman and Co, New York N.Y.). The composition of the synthetic peptides may be confirmed by amino acid analysis or sequencing (e.g., the Edman degradation procedure; Creighton, supra).

Direct synthesis of the agent or variants, homologues, derivatives, fragments or mimetics thereof can be performed using various solid-phase techniques (Roberge J Y et al (1995) Science 269: 202-204) and automated synthesis may be achieved, for example, using the ABI 43 1 A Peptide Synthesizer (Perkin Elmer) in accordance with the instructions provided by the manufacturer. Additionally, the amino acid sequences comprising the agent or any part thereof, may be altered during direct synthesis and/or combined using chemical methods with a sequence from other subunits, or any part thereof, to produce a variant agent or target, such as, for example, a variant oxytocin receptor.

In an alternative embodiment of the invention, the coding sequence of the agent target or variants, homologues, derivatives, fragments or mimetics thereof may be synthesised, in whole or in part, using chemical methods well known in the art (see Caruthers M H et al (1980) Nuc Acids Res Symp Ser 215-23, Horn T et al (1980) Nuc Acids Res Symp Ser 225-232).

### Mimetic

As used herein, the term “mimetic” relates to any chemical which includes, but is not limited to, a peptide, polypeptide, antibody or other organic chemical which has the same qualitative activity or effect as a reference agent, i.e. to a selective oxytocin antagonist for example, to a target, i.e. to a oxytocin receptor for example. That is a mimetic may be a functional equivalent to a known agent.

### Chemical Derivative

The term “derivative” or “derivatised” as used herein includes chemical modification of an agent. Illustrative of such chemical modifications would be replacement of hydrogen by a halo group, an alkyl group, an acyl group or an amino group.

### Chemical Modification

In one embodiment of the present invention, the agent may be a chemically modified agent.

The chemical modification of an agent may either enhance or reduce hydrogen bonding interaction, charge interaction, hydrophobic interaction, Van Der Waals interaction or dipole interaction between the agent and the target.

In one aspect, the identified agent may act as a model (for example, a template) for the development of other compounds.

### Targets

In one aspect of the present invention, an oxytocin receptor may be used as a target in screens to identify agents capable of inhibiting oxytocin receptors. In this regard, the target may comprise an amino acid sequence shown as SEQ ID NO: 1 or a variant, homologue, derivative or fragment thereof which is prepared by recombinant and/or synthetic means or an expression entity comprising same.

In a further aspect of the present invention, both an oxytocin receptor and a vasopressin, particularly V1a, receptor may be used as targets in screens to identify agents capable of selectively inhibiting oxytocin receptors. In this regard, the oxytocin receptor target may comprise an amino acid sequence shown as SEQ ID NO: 1 or a variant, homologue, derivative or fragment thereof which is prepared by recombinant and/or synthetic means or an expression entity comprising same and the vasopressin receptor target may comprise an amino acid sequence shown as SEQ ID NO: 2 or a variant, homologue, derivative or fragment thereof which is prepared by recombinant and/or synthetic means or an expression entity comprising same.

Alternatively, an oxytocin receptor and/or a vasopressin receptor (preferably a V1a receptor) may be used as a target to identify agents capable of mediating an increase in ejaculatory latency through the selective inhibition of the oxytocin receptor. In this respect, the target may be suitable tissue extract.

The target may even be a combination of such tissue and/or recombinant targets.

### Recombinant Methods

The agent and/or target of the present invention may be prepared by recombinant DNA techniques.

In one embodiment, preferably the agent is a selective oxytocin antagonist. The oxytocin antagonist may be prepared by recombinant DNA techniques.

### Amino Acid Sequence

As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “protein”.

The amino acid sequence may be prepared isolated from a suitable source, or it may be made synthetically or it may be prepared by use of recombinant DNA techniques.

In one aspect, the present invention provides an amino acid sequence that is capable of acting as a target (i.e. an oxytocin receptor or a vasopressin, preferably a V1a, receptor) in an assay for the identification of one or more agents and/or derivatives thereof.

In a second aspect, the present invention provides an amino acid sequence that is an agent is capable of selectively inhibiting an oxytocin receptor.

Preferably, the target is an oxytocin receptor.

Preferably, the oxytocin receptor and/or vasopressin, preferably V1a, receptor is an isolated receptor and/or is purified and/or is non-native.

The oxytocin receptor or the vasopressin, preferably V1a, receptor of the present invention may be in a substantially isolated form. It will be understood that the oxytocin receptor or the vasopressin receptor may be mixed with carriers or diluents which will not interfere with the intended purpose of the receptor and/or agent and which will still be regarded as substantially isolated. The oxytocin receptor or the vasopressin receptor of the present invention may also be in a substantially pure form, in which case it will generally comprise the oxytocin receptor or vasopressin receptor in a preparation in which more than 90%, e.g. 95%, 98% or 99% of the oxytocin receptor or vasopressin receptor in the preparation is a peptide having the amino acid sequence shown in SEQ ID NO: 1 or variants, homologues, derivatives or fragments thereof or SEQ ID NO: 2 or variants, homologues, derivatives or fragments thereof, respectively.

### Nucleotide Sequence

As used herein, the term “nucleotide sequence” is synonymous with the term “polynucleotide”.

The nucleotide sequence may be DNA or RNA of genomic or synthetic or of recombinant origin. The nucleotide sequence may be double-stranded or single-stranded whether representing the sense or antisense strand or combinations thereof.

For some applications, preferably, the nucleotide sequence is DNA.

For some applications, preferably, the nucleotide sequence is prepared by use of recombinant DNA techniques (e.g. recombinant DNA).

For some applications, preferably, the nucleotide sequence is cDNA.

For some applications, preferably, the nucleotide sequence may be the same as the naturally occurring form for this aspect.

In one aspect, the present invention provides a nucleotide sequence encoding a substance capable of acting as a target in an assay for the identification of one or more agents and/or derivative thereof.

In one aspect of the present invention the nucleotide sequence encodes an oxytocin receptor.

In a further aspect of the present invention the nucleotide sequence encodes a vasopressin receptor, preferably a V1a receptor.

In one aspect of the present invention, the nucleotide sequence encodes an agent capable of selectively inhibiting oxytocin receptors.

It will be understood by a skilled person that numerous different nucleotide sequences can encode the same target (i.e. oxytocin receptor, such as an oxytocin receptor comprising the amino acid sequence shown in SEQ ID NO: 1, or a vasopressin receptor, such a vasopressin receptor comprising the amino acid sequence shown in SEQ ID NO: 2) as a result of the degeneracy of the genetic code. In addition, it is to be understood that skilled persons may, using routine techniques, make nucleotide substitutions that do not substantially affect the activity encoded by a nucleotide sequence to reflect the codon usage of any particular host organism in which the target is to be expressed. Thus, the terms “variant”, “homologue” or “derivative” in relation to the nucleotide sequence include any substitution of, variation of, modification of, replacement of, deletion of or addition of one (or more) nucleic acid from or to the sequence providing the resultant nucleotide sequence encodes a functional target (i.e. oxytocin receptor for example) according the present invention (or even an agent according to the present invention if said agent comprises a nucleotide sequence or an amino acid sequence).

### Variants/Homologues/Derivatives

In addition to the specific amino acid sequences mentioned herein, the present invention also encompasses the use of variants, homologue and derivatives thereof. Here, the term “homology” can be equated with “identity”.

In the present context, an homologous sequence is taken to include an amino acid sequence which may be at least 75, 85 or 90% identical to the amino acid sequence shown in SEQ ID NO: 1 or SEQ ID NO: 2, preferably at least 95 or 98% identical. In particular, homology should typically be considered with respect to those regions of the sequence known to be essential for an activity. Although homology can also be considered in terms of similarity (i.e. amino acid residues having similar chemical properties/functions), in the context of the present invention it is preferred to express homology in terms of sequence identity.

Homology comparisons can be conducted by eye, or more usually, with the aid of readily available sequence comparison programs. These commercially available computer programs can calculate % homology between two or more sequences.

% homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package (see below) the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12:387). Examples of other software than can perform sequence comparisons include, but are not limited to, the BLAST package (see Ausubel et al., 1999 ibid—Chapter 18), FASTA (Atschul at al., 1990, J. Mol. Biol., 403-410) and the GENEWORKS suite of comparison tools. Both BLAST and FASTA are available for offline and online searching (see Ausubel et al., 1999 ibid, pages 7-58 to 7-60).

However it is preferred to use the GCG Bestfit program. A new tool, called BLAST 2 Sequences is also available for comparing protein and nucleotide sequence (see FEMS Microbiol Lett 1999 174(2): 247-50; FEMS Microbiol Lett 1999 177(1): 187-8 and tatiana@ncbi.nlm.nih.gov).

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). It is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result.

The sequences may also have deletions, insertions or substitutions of amino acid residues which produce a silent change and result in a functionally equivalent substance. Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | PolaR - charged | D E |
| | | K R |
| AROMATIC | | H F W Y |

The present invention also encompasses homologous substitution (substitution and replacement are both used herein to mean the interchange of an existing amino acid residue, with an alternative residue) may occur i.e. like-for-like substitution such as basic for basic, acidic for acidic, polar for polar etc. Non-homologous substitution may also occur i.e. from one class of residue to another or alternatively involving the inclusion of unnatural amino acids such as ornithine (hereinafter referred to as Z), diaminobutyric acid ornithine (hereinafter referred to as B), norleucine ornithine (hereinafter referred to as O), pyriylalanine, thienylalanine, naphthylalanine and phenylglycine.

Replacements may also be made by unnatural amino acids include; alpha* and alpha-disubstituted* amino acids, N-alkyl amino acids*, lactic acid*, halide derivatives of natural amino acids such as trifluorotyrosine*, p-Cl-phenylalanine*, p-Br-phenylalanine*, p-I-phenylalanine*, L-allyl-glycine*, .β-alanine*, L-α-amino butyric acid*, Lγ-amino butyric acid*, L-α-amino isobutyric acid*, L-ε-amino caproic acid^{#}, 7-amino heptanoic acid*, L-methionine sulfone^{#}*, L-norleucine*, L-norvaline*, p-nitro-L-phenylalanine*, L-hydroxyproline#, L-thioproline*, methyl derivatives of phenylalanine (Phe) such as 4-methyl-Phe*, pentamethyl-Phe*, L-Phe (4-amino)^{#}, L-Tyr (methyl)*, L-Phe (4-isopropyl)*, L-Tic (1,2,3,4-tetrahydroisoquinoline-3-carboxyl acid)*, L-diaminopropionic acid^{#} and L-Phe (4-benzyl)*. The notation* has been utilised for the purpose of the discussion above (relating to homologous or non-homologous substitution), to indicate the hydrophobic nature of the derivative whereas # has been utilised to indicate the hydrophilic nature of the derivative, #* indicates amphipathic characteristics.

Variant amino acid sequences may include suitable spacer groups that may be inserted between any two amino acid residues of the sequence including alkyl groups such as methyl, ethyl or propyl groups in addition to amino acid spacers such as glycine or β-alanine residues. A further form of variation, involves the presence of one or more amino acid residues in peptoid form, will be well understood by those skilled in the art. For the avoidance of doubt, “the peptoid form” is used to refer to variant amino acid residues wherein the α-carbon substituent group is on the residue's nitrogen atom rather than the α-carbon. Processes for preparing peptides in the peptoid form are known in the art, for example Simon R J et al., PNAS (1992) 89(20), 9367-9371 and Harwell D C, Trends Biotechnol. (1995) 13(4), 132-134.

### Hybridisation

The term “hybridisation” as used herein shall include “the process by which a strand of nucleic acid joins with a complementary strand through base pairing” as well as the process of amplification as carried out in polymerase chain reaction (PCR) technologies.

Nucleotide sequences capable of selectively hybridising to nucleotide sequences encoding the amino acid sequences of the present invention, or to their complement, will be generally at least 75%, preferably at least 85 or 90% and more preferably at least 95% or 98% homologous to the corresponding complementary nucleotide sequences encoding the amino acid sequences presented herein over a region of at least 20, preferably at least 25 or 30, for instance at least 40, 60 or 100 or more contiguous nucleotides.

The term “selectively hybridizable” means that the nucleotide sequence, when used as a probe, is used under conditions where a target nucleotide sequence is found to hybridise to the probe at a level significantly above background. The background hybridisation may occur because of other nucleotide sequences present, for example, in the cDNA or genomic DNA library being screened. In this event, background implies a level of signal generated by interaction between the probe and a non-specific DNA member of the library which is less than 10 fold, preferably less than 100 fold as intense as the specific interaction observed with the target DNA. The intensity of interaction may be measured, for example, by radiolabelling the probe, e.g. with ³²P.

Hybridisation conditions are based on the melting temperature (Tm) of the nucleic acid binding complex, as taught in Berger and Kimmel (1987, Guide to Molecular Cloning Techniques, Methods in Enzymology, Vol. 152, Academic Press, San Diego Calif.), and confer a defined “stringency” as explained below.

Maximum stringency typically occurs at about Tm-5° C. (5° C. below the Tm of the probe); high stringency at about 5° C. to 10° C. below Tm; intermediate stringency at about 10° C. to 20° C. below Tm; and low stringency at about 20° C. to 25° C. below Tm. As will be understood by those of skill in the art, a maximum stringency hybridisation can be used to identify or detect identical nucleotide sequences while an intermediate (or low) stringency hybridisation can be used to identify or detect similar or related polynucleotide sequences.

In a preferred aspect, the present invention covers nucleotide sequences that can hybridise to a nucleotide sequence encoding an amino acid sequence of the present invention under stringent conditions (e.g. 65° C. and 0.1×SSC (1×SSC=0.15 M NaCl, 0.015 M Na₃ Citrate pH 7.0). Where the nucleotide sequence of the invention is double-stranded, both strands of the duplex, either individually or in combination, are encompassed by the present invention. Where the nucleotide sequence is single-stranded, it is to be understood that the complementary sequence of that nucleotide sequence is also included within the scope of the present invention.

Nucleotide sequences which are not 100% homologous to nucleotide sequences encoding the amino acid sequences of the present invention but fall within the scope of the invention can be obtained in a number of ways. Other variants of the sequences described herein may be obtained for example by probing DNA libraries made from a range of sources. In addition, other viral/bacterial, or cellular homologues particularly cellular homologues found in mammalian cells (e.g. rat, mouse, bovine and primate cells), may be obtained and such homologues and fragments thereof in general will be capable of selectively hybridising to the sequences shown in the sequence listing herein. Such sequences may be obtained by probing cDNA libraries made from or genomic DNA libraries from other animal species, and probing such libraries with probes comprising all or part of the nucleotide sequence set out in herein under conditions of medium to high stringency. Similar considerations apply to obtaining species homologues and allelic variants of the amino acid and/or nucleotide sequences of the present invention.

Variants and strain/species homologues may also be obtained using degenerate PCR which will use primers designed to target sequences within the variants and homologues encoding conserved amino acid sequences within the sequences of the present invention. Conserved sequences can be predicted, for example, by aligning the amino acid sequences from several variants/homologues. Sequence alignments can be performed using computer software known in the art. For example the GCG Wisconsin PileUp program is widely used. The primers used in degenerate PCR will contain one or more degenerate positions and will be used at stringency conditions lower than those used for cloning sequences with single sequence primers against known sequences.

Alternatively, such nucleotide sequences may be obtained by site directed mutagenesis of characterised sequences, such as the nucleotide sequences encoding the amino acid sequences shown in SEQ ID NO: 1 for example. This may be useful where for example silent codon changes are required to sequences to optimise codon preferences for a particular host cell in which the nucleotide sequences are being expressed. Other sequence changes may be desired in order to introduce restriction enzyme recognition sites, or to alter the activity of the protein encoded by the nucleotide sequences.

Nucleotide sequences encoding the amino acid sequences of the present invention may be used to produce a primer, e.g. a PCR primer, a primer for an alternative amplification reaction, a probe e.g. labelled with a revealing label by conventional means using radioactive or non-radioactive labels, or the nucleotide sequences may be cloned into vectors. Such primers, probes and other fragments will be at least 15, preferably at least 20, for example at least 25, 30 or 40 nucleotides in length, and are also encompassed by the term nucleotide sequence of the invention as used herein.

The nucleotide sequences such as a DNA polynucleotides and probes according to the invention may be produced recombinantly, synthetically, or by any means available to those of skill in the art. They may also be cloned by standard techniques.

In general, primers will be produced by synthetic means, involving a stepwise manufacture of the desired nucleic acid sequence one nucleotide at a time. Techniques for accomplishing this using automated techniques are readily available in the art.

Longer nucleotide sequences will generally be produced using recombinant means, for example using a PCR (polymerase chain reaction) cloning techniques.

This will involve making a pair of primers (e.g. of about 15 to 30 nucleotides) flanking a region of the targeting sequence which it is desired to clone, bringing the primers into contact with mRNA or cDNA obtained from an animal or human cell, performing a polymerase chain reaction (PCR) under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector.

Due to the inherent degeneracy of the genetic code, various DNA sequences which encode substantially the same or a functionally equivalent amino acid sequence, may be used to clone and express the target sequences. As will be understood by those of skill in the art, for certain expression systems, it may be advantageous to produce the target sequences with non-naturally occurring codons. Codons preferred by a particular prokaryotic or eukaryotic host (Murray E et al (1989) Nuc Acids Res 17:477-508) can be selected, for example, to increase the rate of the target expression or to produce recombinant RNA transcripts having desirable properties, such as a longer half-life, than transcripts produced from naturally occurring sequence.

### Vector

In one embodiment of the present invention, an agent (i.e. a selective oxytocin antagonist) may be administered directly to an individual.

In another embodiment of the present invention, a vector comprising a nucleotide sequence encoding an agent of the present invention is administered to an individual.

Preferably the recombinant agent is prepared and/or delivered to a target site using a genetic vector.

As it is well known in the art, a vector is a tool that allows or facilitates the transfer of an entity from one environment to another. In accordance with the present invention, and by way of example, some vectors used in recombinant DNA techniques allow entities, such as a segment of DNA (such as a heterologous DNA segment, such as a heterologous cDNA segment), to be transferred into a host and/or a target cell for the purpose of replicating the vectors comprising the nucleotide sequences of the present invention and/or expressing the proteins of the invention encoded by the nucleotide sequences of the present invention. Examples of vectors used in recombinant DNA techniques include but are not limited to plasmids, chromosomes, artificial chromosomes or viruses.

The term “vector” includes expression vectors and/or transformation vectors.

The term “expression vector” means a construct capable of in vivo or in vitro/ex vivo expression.

The term “transformation vector” means a construct capable of being transferred from one species to another.

### Naked DNA

The vectors comprising nucleotide sequences encoding an agent of the present invention for use in treating ejaculatory disorders, in particular premature ejaculation, may be administered directly as “a naked nucleic acid construct”, preferably further comprising flanking sequences homologous to the host cell genome.

As used herein, the term “naked DNA” refers to a plasmid comprising a nucleotide sequences encoding an agent of the present invention together with a short promoter region to control its production. It is called “naked” DNA because the plasmids are not carried in any delivery vehicle. When such a DNA plasmid enters a host cell, such as a eukaryotic cell, the proteins it encodes (such as an agent of the present invention) are transcribed and translated within the cell.

### Non-Viral Delivery

Alternatively, the vectors comprising nucleotide sequences encoding the amino acids of the present invention or an agent of the present invention (i.e. a selective oxytocin antagonist) or a target of the present invention (i.e. an oxytocin receptor) may be introduced into suitable host cells using a variety of non-viral techniques known in the art, such as transfection, transformation, electroporation and biolistic transformation.

As used herein, the term “transfection” refers to a process using a non-viral vector to deliver a gene to a target mammalian cell.

Typical transfection methods include electroporation, DNA biolistics, lipid-mediated transfection, compacted DNA-mediated transfection, liposomes, immunoliposomes, lipofectin, cationic agent-mediated, cationic facial amphiphiles (CFAs) (Nature Biotechnology 1996 14; 556), multivalent cations such as spermine, cationic lipids or polylysine, 1,2-bis(oleoyloxy)-3-(trimethylammonio) propane (DOTAP)-cholesterol complexes (Wolff and Trubetskoy 1998 Nature Biotechnology 16: 421) and combinations thereof.

Uptake of naked nucleic acid constructs by mammalian cells is enhanced by several known transfection techniques for example those including the use of transfection agents. Example of these agents include cationic agents (for example calcium phosphate and DEAE-dextran) and lipofectants (for example Lipofectam™ and Transfectam™). Typically, nucleic acid constructs are mixed with the transfection agent to produce a composition.

### Viral Vectors

Alternatively, the vectors comprising an agent or target of the present invention or nucleotide sequences encoding amino acid sequences of the present invention may be introduced into suitable host cells using a variety of viral techniques which are known in the art, such as for example infection with recombinant viral vectors such as retroviruses, herpes simplex viruses and adenoviruses.

Preferably the vector is a recombinant viral vectors. Suitable recombinant viral vectors include but are not limited to adenovirus vectors, adeno-associated viral (AAV) vectors, herpes-virus vectors, a retroviral vector, lentiviral vectors, baculoviral vectors, pox viral vectors or parvovirus vectors (see Kestler et al 1999 Human Gene Ther 10(10):1619-32). In the case of viral vectors, delivery of the nucleotide sequence encoding the agent of the present invention is mediated by viral/infection of a target cell.

### Targeted Vector

The term “targeted vector” refers to a vector whose ability to infect/transfect/transduce a cell or to be expressed in a host and/or target cell is restricted to certain cell types within the host organism, usually cells having a common or similar phenotype.

### Replication Vectors

Nucleotide sequences encoding an agent (i.e. a selective oxytocin antagonist or, where applicable, a PDEi or PDE5i) of the present invention or a target (such as an oxytocin receptor) may be incorporated into a recombinant replicable vector. The vector may be used to replicate the nucleotide sequence in a compatible host cell. Thus in one embodiment of the present invention, the invention provides a method of making a target of the present invention by introducing a nucleotide sequence of the present invention into a replicable vector, introducing the vector into a compatible host cell, and growing the host cell under conditions which bring about replication of the vector. The vector may be recovered from the host cell.

### Expression Vector

Preferably, an agent of the present invention or a nucleotide sequence encoding an amino acid of the present invention or a target of the present invention which is inserted into a vector is operably linked to a control sequence that is capable of providing for the expression of the coding sequence, such as the coding sequence of the oxytocin receptor of the present invention by the host cell, i.e. the vector is an expression vector. An agent of the present invention or a target produced by a host recombinant cell may be secreted or may be contained intracellularly depending on the sequence and/or the vector used. As will be understood by those of skill in the art, expression vectors containing an agent or target of the present invention coding sequences can be designed with signal sequences which direct secretion of the agent or target of the present invention coding sequences through a particular prokaryotic or eukaryotic cell membrane.

### Expression In Vitro

The vectors of the present invention may be transformed or transfected into a suitable host cell and/or a target cell as described below to provide for expression of an agent or a target of the present invention. This process may comprise culturing a host cell and/or target cell transformed with an expression vector under conditions to provide for expression by the vector of a coding sequence encoding an agent or a target of the present invention and optionally recovering the expressed agent or target of the present invention. The vectors may be for example, plasmid or virus vectors provided with an origin of replication, optionally a promoter for the expression of the said polynucleotide and optionally a regulator of the promoter. The vectors may contain one or more selectable marker genes, for example an ampicillin resistance gene in the case of a bacterial plasmid or a neomycin resistance gene for a mammalian vector. The expression of an agent of the present invention or target of the present invention may be constitutive such that they are continually produced, or inducible, requiring a stimulus to initiate expression. In the case of inducible expression, production of an agent of the present invention or a target can be initiated when required by, for example, addition of an inducer substance to the culture medium, for example dexamethasone or IPTG.

### Fusion Proteins

The oxytocin receptor or vasopressin receptor or an agent (i.e. a selective oxytocin antagonist) of the present invention may be expressed as a fusion protein to aid extraction and purification and/or delivery of the agent of the present invention or the receptor target to an individual and/or to facilitate the development of a screen for agents. Examples of fusion protein partners include glutathione-S-transferase (GST), 6×His, GAL4 (DNA binding and/or transcriptional activation domains) and 6-galactosidase. It may also be convenient to include a proteolytic cleavage site between the fusion protein partner and the protein sequence of interest to allow removal of fusion protein sequences. Preferably the fusion protein will not hinder the activity of the target.

The fusion protein may comprise an antigen or an antigenic determinant fused to the substance of the present invention. In this embodiment, the fusion protein may be a non-naturally occurring fusion protein comprising a substance which may act as an adjuvant in the sense of providing a generalised stimulation of the immune system. The antigen or antigenic determinant may be attached to either the amino or carboxy terminus of the substance.

In another embodiment of the invention, the amino acid sequence may be ligated to a heterologous sequence to encode a fusion protein. For example, for screening of peptide libraries for agents capable of affecting the substance activity, it may be useful to encode a chimeric substance expressing a heterologous epitope that is recognised by a commercially available antibody.

### Host Cells

A wide variety of host cells can be employed for expression of the nucleotide sequences encoding the agent-such as a selective oxytocin antagonist of the present invention—or an oxytocin or vasopressin receptor target of the present invention. These cells may be both prokaryotic and eukaryotic host cells. Suitable host cells include bacteria such as E. coli, yeast, filamentous fungi, insect cells, mammalian cells, typically immortalized, e.g., mouse, CHO, human and monkey cell lines and derivatives thereof.

Examples of suitable expression hosts within the scope of the present invention are fungi such as Aspergillus species (such as those described in EP-A-0184438 and EP-A-0284603) and Trichoderma species; bacteria such as Bacillus species (such as those described in EP-A-0134048 and EP-A-0253455), Streptomyces species and Pseudomonas species; and yeasts such as Kluyveromyces species (such as those described in EP-A-0096430 and EP-A-0301670) and Saccharomyces species. By way of example, typical expression hosts may be selected from Aspergillus niger, Aspergillus niger var. tubigenis, Aspergillus niger var. awamori, Aspergillus aculeatis, Aspergillus nidulans, Aspergillus oryzae, Trichoderma reesei, Bacillus subtilis, Bacillus licheniformis, Bacillus amyloliquefaciens, Kluyveromyces lactis and Saccharomyces cerevisiae.

The use of suitable host cells—such as yeast, fungal and plant host cells—may provide for post-translational modifications (e.g. myristoylation, glycosylation, truncation, lapidation and tyrosine, serine or threonine phosphorylation) as may be needed to confer optimal biological activity on recombinant expression products of the present invention.

Preferred host cells are able to process the expression products to produce an appropriate mature polypeptide. Examples of processing includes but is not limited to glycosylation, ubiquitination, disulfide bond formation and general post-translational modification.

### Antibodies

In one embodiment of the present invention, the agent may be an antibody. In addition, or in the alternative, the target may be an antibody.

Antibodies may be produced by standard techniques, such as by immunisation with the substance of the invention or by using a phage display library.

For the purposes of this invention, the term “antibody”, unless specified to the contrary, includes but is not limited to, polyclonal, monoclonal, chimeric, single chain, Fab fragments, fragments produced by a Fab expression library, as well as mimetics thereof. Such fragments include fragments of whole antibodies which retain their binding activity for a target substance, Fv, F(ab′) and F(ab′)₂ fragments, as well as single chain antibodies (scFv), fusion proteins and other synthetic proteins which comprise the antigen-binding site of the antibody. Furthermore, the antibodies and fragments thereof may be humanised antibodies. Neutralising antibodies, i.e., those which inhibit biological activity of the substance polypeptides, are especially preferred for diagnostics and therapeutics.

If polyclonal antibodies are desired, a selected mammal (e.g., mouse, rabbit, goat, horse, etc.) is immunised with an immunogenic polypeptide bearing a epitope(s) obtainable from an identified agent and/or substance of the present invention. Depending on the host species, various adjuvants may be used to increase immunological response. Such adjuvants include, but are not limited to, Freund's, mineral gels such as aluminium hydroxide, and surface active substances such as lysolecithin, pluronic polyols, polyanions, peptides, oil emulsions, keyhole limpet hemocyanin, and dinitrophenol. BCG (Bacilli Calmette-Guerin) and Corynebacterium parvum are potentially useful human adjuvants which may be employed if purified the substance polypeptide is administered to immunologically compromised individuals for the purpose of stimulating systemic defense.

Serum from the immunised animal is collected and treated according to known procedures. If serum containing polyclonal antibodies to an epitope obtainable from an identified agent and/or substance of the present invention contains antibodies to other antigens, the polyclonal antibodies can be purified by immunoaffinity chromatography. Techniques for producing and processing polyclonal antisera are known in the art. In order that such antibodies may be made, the invention also provides polypeptides of the invention or fragments thereof haptenised to another polypeptide for use as immunogens in animals or humans.

Monoclonal antibodies directed against epitopes obtainable from an identified agent and/or substance of the present invention can also be readily produced by one skilled in the art. The general methodology for making monoclonal antibodies by hybridomas is well known. Immortal antibody-producing cell lines can be created by cell fusion, and also by other techniques such as direct transformation of B lymphocytes with oncogenic DNA, or transfection with Epstein-Barr virus. Panels of monoclonal antibodies produced against orbit epitopes can be screened for various properties; i.e., for isotype and epitope affinity.

Monoclonal antibodies to the substance and/or identified agent may be prepared using any technique which provides for the production of antibody molecules by continuous cell lines in culture. These include, but are not limited to, the hybridoma technique originally described by Koehler and Milstein (1975 Nature 256:495-497), the human B-cell hybridoma technique (Kosbor et al (1983) Immunol Today 4:72; Cote et al (1983) Proc Natl Acad Sci 80:2026-2030) and the EBV-hybridoma technique (Cole et al (1985) Monoclonal Antibodies and Cancer Therapy, Alan R Liss Inc, pp 77-96). In addition, techniques developed for the production of “chimeric antibodies”, the splicing of mouse antibody genes to human antibody genes to obtain a molecule with appropriate antigen specificity and biological activity can be used (Morrison et al (1984) Proc Natl Acad Sci 81:6851-6855; Neuberger et al (1984) Nature 312:604-608; Takeda et al (1985) Nature 314:452-454). Alternatively, techniques described for the production of single chain antibodies (U.S. Pat. No. 4,946,779) can be adapted to produce the substance specific single chain antibodies.

Antibodies, both monoclonal and polyclonal, which are directed against epitopes obtainable from an identified agent and/or substance are particularly useful in diagnosis, and those which are neutralising are useful in passive immunotherapy. Monoclonal antibodies, in particular, may be used to raise anti-idiotype antibodies. Anti-idiotype antibodies are immunoglobulins which carry an “internal image” of the substance and/or agent against which protection is desired. Techniques for raising anti-idiotype antibodies are known in the art. These anti-idiotype antibodies may also be useful in therapy.

Antibodies may also be produced by inducing in vivo production in the lymphocyte population or by screening recombinant immunoglobulin libraries or panels of highly specific binding reagents as disclosed in Orlandi et al (1989, Proc Natl Acad Sci 86: 3833-3837), and Winter G and Milstein C (1991; Nature 349:293-299).

Antibody fragments which contain specific binding sites for the substance may also be generated. For example, such fragments include, but are not limited to, the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed to allow rapid and easy identification of monoclonal Fab fragments with the desired specificity (Huse W D et al (1989) Science 256:1275-128 1).

### Reporters

A wide variety of reporters may be used in the assay methods (as well as screens) of the present invention with preferred reporters providing conveniently detectable signals (e.g. by spectroscopy). By way of example, a reporter gene may encode an enzyme which catalyses a reaction which alters light absorption properties.

Examples of reporter molecules include but are not limited to β-galactosidase, invertase, green fluorescent protein, luciferase, chloramphenicol, acetyltransferase, β-glucuronidase, exo-glucanase and glucoamylase. Alternatively, radiolabelled or fluorescent tag-labelled nucleotides can be incorporated into nascent transcripts which are then identified when bound to oligonucleotide probes.

In one preferred embodiment, the production of the reporter molecule is measured by the enzymatic activity of the reporter gene product, such as β-galactosidase.

A variety of protocols for detecting and measuring the expression of the target, such as by using either polyclonal or monoclonal antibodies specific for the protein, are known in the art. Examples include enzyme-linked immunosorbent assay (ELISA), radioimmunoassay (RIA) and fluorescent activated cell sorting (FACS). A two-site, monoclonal-based immunoassay utilising monoclonal antibodies reactive to two non-interfering epitopes on polypeptides is preferred, but a competitive binding assay may be employed. These and other assays are described, among other places, in Hampton R et al (1990, Serological Methods, A Laboratory Manual, APS Press, St Paul Minn.) and Maddox D E et al (1983, J Exp Med 15 8:121 1).

A wide variety of labels and conjugation techniques are known by those skilled in the art and can be used in various nucleic and amino acid assays. Means for producing labelled hybridisation or PCR probes for detecting the target polynucleotide sequences include oligolabelling, nick translation, end-labelling or PCR amplification using a labelled nucleotide. Alternatively, the coding sequence, or any portion of it, may be cloned into a vector for the production of an mRNA probe. Such vectors are known in the art, are commercially available, and may be used to synthesise RNA probes in vitro by addition of an appropriate RNA polymerase such as T7, T3 or SP6 and labelled nucleotides.

A number of companies such as Pharmacia Biotech (Piscataway, N.J.), Promega (Madison, Wis.), and US Biochemical Corp (Cleveland, Ohio) supply commercial kits and protocols for these procedures. Suitable reporter molecules or labels include those radionuclides, enzymes, fluorescent, chemiluminescent, or chromogenic agents as well as substrates, cofactors, inhibitors, magnetic particles and the like. Patents teaching the use of such labels include U.S. Pat. No. 3,817,837; U.S. Pat. No. 3,850,752; U.S. Pat. No. 3,939,350; U.S. Pat. No. 3,996,345; U.S. Pat. No. 4,277,437; U.S. Pat. No. 4,275,149 and U.S. Pat. No. 4,366,241.

Also, recombinant immunoglobulins may be produced as shown in U.S. Pat. No. 4,816,567.

Additional methods to quantify the expression of a particular molecule include radiolabeling (Melby P C et a/1993 J Immunol Methods 159:235-44) or biotinylating (Duplaa C et al 1993 Anal Biochem 229-36) nucleotides, coamplification of a control nucleic acid, and standard curves onto which the experimental results are interpolated. Quantification of multiple samples may be speeded up by running the assay in an ELISA format where the oligomer of interest is presented in various dilutions and a spectrophotometric or calorimetric response gives rapid quantification.

Although the presence/absence of marker gene expression suggests that the gene of interest is also present, its presence and expression should be confirmed. For example, if the nucleotide sequence is inserted within a marker gene sequence, recombinant cells containing the same may be identified by the absence of marker gene function. Alternatively, a marker gene can be placed in tandem with a target coding sequence under the control of a single promoter. Expression of the marker gene in response to induction or selection usually indicates expression of the target as well.

Alternatively, host cells which contain a coding sequence for the target and express the target coding regions may be identified by a variety of procedures known to those of skill in the art. These procedures include, but are not limited to, DNA-DNA or DNA-RNA hybridisation and protein bioassay or immunoassay techniques which include membrane-based, solution-based, or chip-based technologies for the detection and/or quantification of the nucleic acid or protein.

### Screens

Any one or more of an appropriate target—such as an oxytocin receptor and/or a vasopressin, preferably a V1a, receptor-may be used for identifying an agent, e.g. a selective oxytocin receptor antagonist, in any of a variety of drug screening techniques. The target employed in such a test may be free in solution, affixed to a solid support, borne on a cell surface, or located intracellularly. The target may even be within an animal model, wherein said target may be an exogenous target or an introduced target. The animal model will be a non-human animal model. The abolition of target activity or the formation of binding complexes between the target and the agent being tested may be measured.

Techniques for drug screening may be based on the method described in Geysen, European Patent Application 84/03564, published on Sep. 13, 1984. In summary, large numbers of different small peptide test compounds are synthesised on a solid substrate, such as plastic pins or some other surface. The peptide test compounds are reacted with a suitable target or fragment thereof and washed. Bound entities are then detected—such as by appropriately adapting methods well known in the art. A purified target can also be coated directly onto plates for use in a drug screening techniques. Alternatively, non-neutralising antibodies can be used to capture the peptide and immobilise it on a solid support.

This invention also contemplates the use of competitive drug screening assays in which neutralising antibodies capable of binding a target specifically compete with a test compound for binding to a target.

Another technique for screening provides for high throughput screening (HTS) of agents having suitable binding affinity to the substances and is based upon the method described in detail in WO 84/03564.

It is expected that the assay methods of the present invention will be suitable for both small and large-scale screening of test compounds as well as in quantitative assays.

In a preferred aspect, the screen of the present invention comprises at least the following steps (which need not be in this same consecutive order): (a) conducting an in vitro screen to determine whether a candidate agent has the relevant activity (such as modulation of an oxytocin receptor); (b) conducting one or more selectivity screens to determine the selectivity of said candidate agent (e.g. to see if said agent is also a vasopressin, particularly V1a, receptor inhibitor)-such as by using the assay protocol presented herein; and (c) conducting an in vivo screen with said candidate agent (e.g. using a functional animal model, including determining the selectivity of the agent by determining the effect of the agent on vasopressin, particularly V1a, receptors). Typically, if said candidate agent passes screen (a) and screen (b) then screen (c) is performed.

### Diagnostic Methods/Compositions/Kits

The present invention also provides a diagnostic method, composition or kit for the detection of a pre-disposition for premature ejaculation. In this respect, the method, composition or kit will comprise means for detecting an entity, preferably oxytocin, in a test sample, preferably a blood sample taken from a sexually aroused male.

In order to provide basis for the diagnosis of premature ejaculation, normal or standard values for an entity should be established. This may be accomplished by combining body fluids taken from normal subjects, either animal or human, taken at various time periods following sexual arousal, with an antibody to the entity under conditions suitable for complex formation which are well known in the art. The amount of standard complex formation may be quantified by comparing it to a dilution series of positive controls where a known amount of antibody is combined with known concentrations of a purified target. Then, standard values obtained from normal samples may be compared with values obtained from samples from subjects potentially affected by premature ejaculation. Deviation between standard and subject values establishes the presence of the disease state.

The entity itself, or any part thereof, may provide the basis for a diagnostic and/or therapeutic compound. For diagnostic purposes, target polynucleotide sequences may be used to detect and quantify gene expression in conditions, disorders or diseases in which premature ejaculation may be implicated.

The target encoding polynucleotide sequence may be used for the diagnosis of premature ejaculation resulting from expression of the target. For example, polynucleotide sequences encoding an entity may be used in hybridisation or PCR assays of tissues from biopsies or autopsies or biological fluids, to detect expression of the entity. The form of such qualitative or quantitative methods may include Southern or northern analysis, dot blot or other membrane-based technologies; PCR technologies; dip stick, pin or chip technologies; and ELISA or other multiple sample formal technologies. All of these techniques are well known in the art and are in fact the basis of many commercially available diagnostic kits.

Such assays may be tailored to evaluate the efficacy of a particular therapeutic treatment regime and may be used in animal studies, in clinical trials, or in monitoring the treatment of an individual. If disease is established, an existing therapeutic agent is administered, and treatment profile or values may be generated. Finally, the assay may be repeated on a regular basis to evaluate whether the values progress toward or return to the normal or standard pattern. Successive treatment profiles may be used to show the efficacy of treatment over a period of several days or several months.

### Assay Methods

The assay methods according to the present invention may use one or more of the following techniques which include but are not limited to; competitive and non-competitive assays, radioimmunoassay, bioluminescence and chemiluminescence assays, fluorometric assays, sandwich assays, immunoradiometric assays, dot blots, enzyme linked assays including ELISA, microtiter plates, antibody coated strips or dipsticks for rapid monitoring of urine or blood, immunohistochemistry and immunocytochemistry.

### Probes

Another aspect of the subject invention is the provision of nucleic acid hybridisation or PCR probes which are capable of detecting (especially those that are capable of selectively selecting) polynucleotide sequences, including genomic sequences, encoding a target coding region, such as a region encoding an oxytocin receptor, or closely related molecules, such as alleles. The specificity of the probe, i.e., whether it is derived from a highly conserved, conserved or non-conserved region or domain, and the stringency of the hybridisation or amplification (high, intermediate or low) will determine whether the probe identifies only naturally occurring target coding sequence, or related sequences. Probes for the detection of related nucleic acid sequences are selected from conserved or highly conserved nucleotide regions of target family members and such probes may be used in a pool of degenerate probes. For the detection of identical nucleic acid sequences, or where maximum specificity is desired, nucleic acid probes are selected from the non-conserved nucleotide regions or unique regions of the target polynucleotides. As used herein, the term “non-conserved nucleotide region” refers to a nucleotide region that is unique to a target coding sequence disclosed herein and does not occur in related family members.

PCR as described in U.S. Pat. No. 4,683,195, U.S. Pat. No. 4,800,195 and U.S. Pat. No. 4,965,188 provides additional uses for oligonucleotides based upon target sequences. Such oligomers are generally chemically synthesised, but they may be generated enzymatically or produced from a recombinant source. Oligomers generally comprise two nucleotide sequences, one with sense orientation (5′->3′) and one with antisense (3′<-5′) employed under optimised conditions for identification of a specific gene or condition. The same two oligomers, nested sets of oligomers, or even a degenerate pool of oligomers may be employed under less stringent conditions for detection and/or quantification of closely related DNA or RNA sequences.

The nucleic acid sequence for an agent or a target can also be used to generate hybridisation probes as previously described, for mapping the endogenous genomic sequence. The sequence may be mapped to a particular chromosome or to a specific region of the chromosome using well known techniques. These include in situ hybridisation to chromosomal spreads (Verma et al (1988) Human Chromosomes: A Manual of Basic Techniques, Pergamon Press, New York City), flow-sorted chromosomal preparations, or artificial chromosome constructions such as YACs, bacterial artificial chromosomes (BACs), bacterial PI constructions or single chromosome cDNA libraries.

In situ hybridisation of chromosomal preparations and physical mapping techniques such as linkage analysis using established chromosomal markers are invaluable in extending genetic maps. Examples of genetic maps can be found in Science (1995; 270:410f and 1994; 265:1981f). Often the placement of a gene on the chromosome of another mammalian species may reveal associated markers even if the number or arm of a particular human chromosome is not known. New sequences can be assigned to chromosomal arms, or parts thereof, by physical mapping. This provides valuable information to investigators searching for disease genes using positional cloning or other gene discovery techniques. Once a disease or syndrome has been crudely localised by genetic linkage to a particular genomic region any sequences mapping to that area may represent associated or regulatory genes for further investigation. The nucleotide sequence of the subject invention may also be used to detect differences in the chromosomal location due to translocation, inversion, etc. between normal, carrier or affected individuals.

### Organism

The term “organism” in relation to the present invention includes any organism that could comprise the target and/or products obtained therefrom. Examples of organisms may include a mammal, a fungus, yeast or a plant.

The term “transgenic organism” in relation to the present invention includes any organism that comprises the target and/or products obtained therefrom.

### Transformation of Host Cells/Host Organisms

As indicated earlier, the host organism can be a prokaryotic or a eukaryotic organism. Examples of suitable prokaryotic hosts include E. coli and Bacillus subtilis. Teachings on the transformation of prokaryotic hosts is well documented in the art, for example see Sambrook et al (Molecular Cloning: A Laboratory Manual, 2nd edition, 1989, Cold Spring Harbor Laboratory Press) and Ausubel et al., Current Protocols in Molecular Biology (1995), John Wiley & Sons, Inc.

If a prokaryotic host is used then the nucleotide sequence may need to be suitably modified before transformation-such as by removal of introns.

In another embodiment the transgenic organism can be a yeast. In this regard, yeast have also been widely used as a vehicle for heterologous gene expression.

The species Saccharomyces cerevisiae has a long history of industrial use, including its use for heterologous gene expression. Expression of heterologous genes in Saccharomyces cerevisiae has been reviewed by Goodey et al (1987, Yeast Biotechnology, D R Berry et al, eds, pp 401-429, Allen and Unwin, London) and by King et al (1989, Molecular and Cell Biology of Yeasts, E F Walton and G T Yarronton, eds, pp 107-133, Blackie, Glasgow).

For several reasons Saccharomyces cerevisiae is well suited for heterologous gene expression. First, it is non-pathogenic to humans and it is incapable of producing certain endotoxins. Second, it has a long history of safe use following centuries of commercial exploitation for various purposes. This has led to wide public acceptability. Third, the extensive commercial use and research devoted to the organism has resulted in a wealth of knowledge about the genetics and physiology as well as large-scale fermentation characteristics of Saccharomyces cerevisiae.

A review of the principles of heterologous gene expression in Saccharomyces cerevisiae and secretion of gene products is given by E Hinchcliffe E Kenny (1993, “Yeast as a vehicle for the expression of heterologous genes”, Yeasts, Vol. 5, Anthony H Rose and J Stuart Harrison, eds, 2nd edition, Academic Press Ltd.).

Several types of yeast vectors are available, including integrative vectors, which require recombination with the host genome for their maintenance, and autonomously replicating plasmid vectors.

In order to prepare the transgenic Saccharomyces, expression constructs are prepared by inserting the nucleotide sequence of the present invention into a construct designed for expression in yeast. Several types of constructs used for heterologous expression have been developed. The constructs contain a promoter active in yeast fused to the nucleotide sequence of the present invention, usually a promoter of yeast origin, such as the GAL1 promoter, is used. Usually a signal sequence of yeast origin, such as the sequence encoding the SUC2 signal peptide, is used. A terminator active in yeast ends the expression system.

For the transformation of yeast several transformation protocols have been developed. For example, a transgenic Saccharomyces according to the present invention can be prepared by following the teachings of Hinnen et al (1978, Proceedings of the National Academy of Sciences of the USA 75, 1929); Beggs, J D (1978, Nature, London, 275, 104); and Ito, H et al (1983, J Bacteriology 153, 163-168).

The transformed yeast cells are selected using various selective markers. Among the markers used for transformation are a number of auxotrophic markers such as LEU2, HIS4 and TRP1, and dominant antibiotic resistance markers such as aminoglycoside antibiotic markers, e.g. G418.

Another host organism is a plant. The basic principle in the construction of genetically modified plants is to insert genetic information in the plant genome so as to obtain a stable maintenance of the inserted genetic material. Several techniques exist for inserting the genetic information, the two main principles being direct introduction of the genetic information and introduction of the genetic information by use of a vector system. A review of the general techniques may be found in articles by Potrykus (Annu Rev Plant Physiol Plant Mol Biol [1991] 42:205-225) and Christou (Agro-Food-Industry Hi-Tech March/April 1994 17-27). Further teachings on plant transformation may be found in EP-A-0449375.

Thus, the present invention also provides a method of transforming a host cell with a nucleotide sequence that is to be the target or is to express the target. Host cells transformed with the nucleotide sequence may be cultured under conditions suitable for the expression and recovery of the encoded protein from cell culture. The protein produced by a recombinant cell may be secreted or may be contained intracellularly depending on the sequence and/or the vector used. As will be understood by those of skill in the art, expression vectors containing coding sequences can be designed with signal sequences which direct secretion of the coding sequences through a particular prokaryotic or eukaryotic cell membrane. Other recombinant constructions may join the coding sequence to nucleotide sequence encoding a polypeptide domain which will facilitate purification of soluble proteins (Kroll D J et al (1993) DNA Cell Biol 12:441-53).

### PDE Inhibitor—Test Methods

PDE action potency values referred to herein are determined by the following assays:

### Phosphodiesterase (PDE) Inhibitory Activity

Preferred PDE compounds suitable for use in accordance with the present invention are potent and selective cGMP PDE5 inhibitors. In vitro PDE inhibitory activities against cyclic guanosine 3′,5′-monophosphate (cGMP) and cyclic adenosine 3′,5′-monophosphate (cAMP) phosphodiesterases can be determined by measurement of their IC₅₀ values (the concentration of compound required for 50% inhibition of enzyme activity).

The required PDE enzymes can be isolated from a variety of sources, including human corpus cavernosum, human and rabbit platelets, human cardiac ventricle, human skeletal muscle and bovine retina, essentially by the method of W. J. Thompson and M. M. Appleman (Biochem., 1971, 10, 311). In particular, the cGMP-specific PDE (PDE5) and the cGMP-inhibited cAMP PDE (PDE3) can be obtained from human corpus cavernosum tissue, and the cAMP-specific PDE (PDE4) from human skeletal muscle. Phosphodiesterases 7-11 can be generated from full length human recombinant clones transfected into SF9 cells.

Assays can be performed either using a modification of the “batch” method of W. J. Thompson et al. (Biochem., 1979, 18, 5228) or using a scintillation proximity assay for the direct detection of AMP/GMP using a modification of the protocol described by Amersham plc under product code TRKQ7090/7100. In summary, the effect of PDE inhibitors was investigated by assaying a fixed amount of enzyme in the presence of varying inhibitor concentrations and low substrate, (cGMP or cAMP in a 3:1 ratio unlabelled to [³H]-labeled at a conc˜⅓ Kₘ) such that IC₅₀≅Kᵢ. The final assay volume was made up to 100 μl with assay buffer [20 mM Tris-HCl pH 7.4, 5 mM MgCl₂, 1 mg/ml bovine serum albumin]. Reactions were initiated with enzyme, incubated for 30-60 min at 30° C. to give <30% substrate turnover and terminated with 50 μl yttrium silicate SPA beads (containing 3 mM of the respective unlabelled cyclic nucleotide for PDEs 9 and 11). Plates were re-sealed and shaken for 20 min, after which the beads were allowed to settle for 30 min in the dark and then counted on a TopCount plate reader (Packard, Meriden, Conn.) Radioactivity units were converted to % activity of an uninhibited control (100%), plotted against inhibitor concentration and inhibitor IC₅₀ values obtained using the ‘Fit Curve’ Microsoft Excel extension.

### Functional Activity

This can be assessed in vitro by determining the capacity of a compound of the invention to enhance sodium nitroprusside-induced relaxation of pre-contracted rabbit corpus cavernosum tissue strips, as described by S. A. Ballard et al. (Brit. J. Pharmacol., 1996, 118 (suppl.), abstract 153P).

The invention will now be further described only by way of example in which reference is made to the following Figures and List of Sequences:

### FIGURES

FIG. 1 shows a graph which depicts the effect of an oxytocin receptor antagonist L-368,899 on p-chloroamphetamine (PCA)-induced ejaculation in anaesthetised rats; and

FIG. 2 shows a graph depicting the effect of a selective oxytocin antagonist (L-368,899) on seminal vesicle pressure in anaesthetised rats.

### LIST OF SEQUENCES

SEQ ID NO: 1 shows an amino sequence for human oxytocin receptor; and

SEQ ID NO: 2 shows an amino acid sequence for human vasopressin VIA receptor.

### EXAMPLES

### 1.0 Methods

### 1.1. Animal Test Method

### 1.1.1. Penile Erection and Ejaculation Test Method in Anaesthetised Rats

In order to study penile erection and ejaculation the method used was based on the methodology taught in Yonezawa et al (2000) Life Sciences 67, 3031-3039. For ease of reference, this methodology is recited below:

Male Wistar-ST strain rats, weighing 350-450 g, are used. Prior to the experiments the animals are housed in groups (2 rats per cage) under controlled 12 h light-dark cycle (lights on at 07:00), constant temperature (23±1° C.) and humidity (55±5%). They have free access to standard food pellets and water.

Rats are anesthestised with sodium pentobarbital (50 mg/kg, i.p.) and are placed in the supine position. The penis is extruded from its sheath and gently held by a wooden applicator positioned at the base of the penis. The test compounds are administered i.p. immediately before the sheath retraction and the penile responses, including penile erection, redding and expansion of the penile body, glans erection, engorgement and slight flaring of the glans and cup, glans erection with intense flaring of the glans, are recorded. Latencies from test compound administration to the initial penile response and ejaculation were also measured.

The effect of a test compound on PCA induced ejaculation is also assessed by weighing the ejaculates accumulated over 30 mins. A suitable method using conscious rats is described in Renyi (1985) Neuropharmacology, Vol. 24. No. 8, pp 697-704.

Intracavernosal pressure is also determined in rats anesthetised with sodium pentobarbitol (50 mg/kg, i.p.). Further small additional amounts (5 mg) may be injected throughout the experimental period as required. The penis is extruded from its sheath and the intracavernosal pressuer (ICP) was measured by inserting a stainless steel needle (23-gauge) into one corpus cavernosum. The needle is attached to a heparinized saline (10 U/ml)-filled teflon tube and connected to a pressure transducer (NEC-San-Ei 7500).

### 1.1.2 Male Sexual Behaviour Model:

For all the sexual behaviour tests, the male rats were placed in an observation arena (50-60 cm diameter), starting 5 hours into the dark cycle and observed under red illumination. Three to four minutes after placing the male in the arena, a receptive female (ovariectomised, oestradiol benzoate/progesterone injection 48 hour before behavioural study) was introduced into the arena and the following parameters noted:
i) ejaculatory latency (EJL; time taken from addition of receptive female into the arena to ejaculation);
ii) copulatory efficiency (CE; ejaculatory latency/the number of intromissions to ejaculation, i.e. the number of seconds between intromissions);
iii) intromission frequency (IF; the number of intromissions to ejaculation);
iv) mount frequency (MF; the number of mounts to ejaculation);
v) post ejaculatory interval (PEI; the time taken from ejaculation to the commencement of copulatory behaviour).

### 2.0 Selective Oxytocin Receptor Antagonists

The compound used in the following Examples was as follows:

Selective oxytocin receptor antagonist L-368,899. Further details regarding this compound are provided hereinabove. L-368,899 is more than 20-fold selective towards oxytocin receptors as compared with V1a receptors [6.3 nM OT: 148 nM V1a].

### Example 1

### Delaying Ejaculation in the Presence of a Selective Oxvtocin Receptor Antagonist (L-368,899)

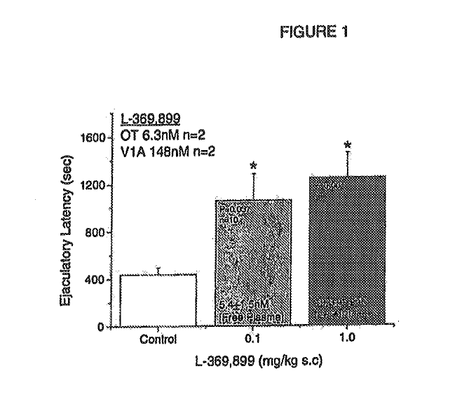

An oxytocin receptor antagonist L-368,899 significantly delayed p-chloroamphetamine (PCA)-induced ejaculation at oxytocin selective doses in anaesthetised rats (0.1-10 mgkg⁻¹ sc). Ejaculation was delayed 140% (near maximal effect) at free plasma concentrations 5.4±1.5 nM (0.9×Ki OT, see FIG. 1)—it has been assumed that at this does any activity arises from antagonism of oxytocin receptors.

Erectogenic mechanisms were largely unaffected by oxytocin receptor blockade—the number of penile cups and flares was similar in control and oxytocin antagonist studies (see Table 1 below). Following a 1 mgkg⁻¹ sc dose of L-368,899 (a dose that significantly delays ejaculation)—95% of PCA-induced erections resulted in penile cups compare to 94% in vehicle control groups and 61% of PCA-induced erections resulted in penile flares compare to 63% in vehicle control groups.

TABLE 1

L-368,899

| | Vehicle | 0.1 mg/kg | 1.0 mg/kg |
|---|---|---|---|
| Penile | 94% | 98% | 95% |
| Cups | | | |
| Penile | 63% | 88% | 61% |
| Flares | | | |

L-368,899 has very poor CNS penetration and as such this study shows that oxytocin has a peripheral site of action in PCA-induced ejaculation. PCA is a 5HT releaser which activates non-adrenergic non-cholinergic nerves that produce penile erection and the sympathetic pathways that control ejaculation. These prosexual effects are thought to be mediated via release of spinal 5HT acting on 5HT1B and 5HT2C receptors. PCA also induces the secretion of oxytocin—possibly from the posterior pituitary or from spinal centres. This increase in oxytocin, like in man, is involved in the ejaculatory process since antagonism of oxytocin receptors in these studies has significant effects on the time taken to achieve ejaculation.

Using a rodent model of ejaculatory, that reflects human ejaculatory physiology, we have shown that that peripheral oxytocin receptors are involved in the ejaculatory mechanisms. These effects could be direct or via modulation of sympathetic innervation of the internal reproductive organs. We can not discount a role for central oxytocin receptors. More over, the study shows that an oxytocin antagonist will be useful in the treatment of premature ejaculation by delaying ejaculation.

### Example 2

### Effect of a Selective Oxytocin Antagonist (L-368,899) on Seminal Vesicle Pressure in Anaesthetised Rats

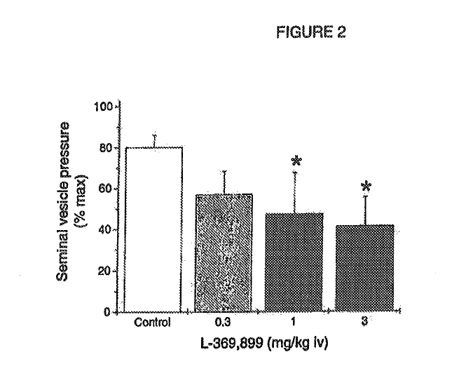

L-368,899 significantly reduced splanchnic nerve-stimulated increases in seminal vesicle pressure in anaesthetised rats (1-3 mgkg⁻¹ iv). Seminal vesicle contraction is essential for emission and the seminal fluid delivered into the prostatic urethra is thought to trigger ejaculation. Oxytocin has direct contractile effects on mammalian seminal vesicles and may additionally have a neuromodulator role influencing sympathetic innervation during ejaculation. In this study seminal vesicle contraction was reduced by 41% after a 1 mgkg⁻¹ bolus injection (see FIG. 2 below). Preliminary studies suggest that the free plasma concentration for L-368,899 achieved after a 1.0 mgkg⁻¹ iv injection is approximately 60 nM—based on literature PK and protein plasma binding.

The data suggests that oxytocin is released during splanchnic nerve stimulation and that the peptide plays a physiological role in the generation of intravesicular pressure and in the process of emission prior to ejaculation. This study supports a role for systemic oxytocin influencing the peripheral ejaculatory response-these effects may be direct or via modulation of sympathetic innervation of the seminal vesicle. Oxytocin may be modulating the contraction of ducts and glandular lobules throughout the male genital tract thus influencing the fluid volume of different ejaculate components. Increasing ejaculate volume is thought to shorten the time from intromission to ejaculation and hence an oxytocin antagonist could be useful in the treatment of premature ejaculation by delayed seminal emission.

### Example 3

### Effect of a Selective Oxytocin Antagonist (L-368,899) on Copulatory Behaviour in Rats

L-368,899 has no effect on copulatory behaviour in sexually-experienced rats at doses up to 10 mgkg⁻¹ sc. Rodent copulatory behaviour is characterised by a series of mounts, with and without vaginal insertion (50-80% of mounts result in intromission [vaginal penetration]) and ejaculation occurs after 6 to 12 intromissions. Each intromission lasts a matter of seconds—it is not possible to quantify intromission length i.e. intravaginal latency. The effect of L-368,899 was assessed on a number of copulatory parameters (see above). We have focused copulatory efficiency as a measure that summarises vaginal penetration.

There were no effects of L-368,899 on copulatory efficiency at any of the doses tested (0.05-10 mgkg⁻¹ sc, see Table 2 below). Preliminary pharmacokinetic studies suggest that 30 minutes after a 1 mgkg⁻¹ sc and a 10 mgkg⁻¹ sc injection, a free plasma concentration of 4.5 nM and 40 nM would be expected respectively.

TABLE 2

| | | L-368,899 | L-368,899 |
|---|---|---|---|
| | Vehicle | 1 mgkg⁻¹ sc | 10 mgkg⁻¹ sc |
| Copulatory | 7.0E−2 i.e. | 6.2E−2 i.e. | 7.0E−2 i.e. |
| efficiency | 14 s/intromission | 16 s/intromission | 14 s/intromission |

mean ± sem (n = 14, 8, 5 respectively).

L-368,899 was also administered intracerebroventricularly (icv). L-368,899 has no significant effects on copulatory efficiency in sexually-experienced rats when dosed 50 ng/rat icy.

| | | L-368,899 |
|---|---|---|
| | Vehicle | 50 ng/rat icv |
| Copulatory efficiency | 3.9E−2 i.e. | 3.4E−2 i.e. |
| | 25 s/intromission | 29 s/intromission |

mean ± sem (n = 4). # p = 0.057

### Example 4

### Effect of a Non-Selective Oxytocin Antagonist (Vasotocin) on Copulatory Behaviour

Previous studies investigating the effects of d(CH₂)₅Tyr(Me)-Orn⁸-vasotocin, a peptidergic non-selective oxytocin antagonist, on copulatory behaviour showed that at doses up to 25 ng/rat icy there was no effect of ejaculatory latency. After a 5 ng/rat icy injection there was a (significant) decrease in copulatory efficiency-46 sec/intromission compared to 25 sec/intromission in vehicle control animals. This effect maybe mediated by oxytocin and/or vasopressin receptors. After 50 ng/rat icy administration copulatory behaviour was abolished (Melis et al Neuroscience Letters 265 (1999) 171-174) demonstrated that 100 ng/rat icy reduces the number of non-contact erections 100 ng/rat icy abolished drug-induced erections completely. This latter data, in contrast to studies with a selective oxytocin receptor antagonist (L-368,899), may indicate that antagonism of both central oxytocin and vasopressin receptors could be detrimental to sexual behaviour including erectile mechanisms and arousability.

### ABBREVIATIONS

PE=Premature ejaculation
OT=oxytocin
cAMP=cyclic adenosine-3′,5′-monophosphate
cGMP=cyclic guanosine-3′,5′-monophosphate
PDE=phosphodiesterase
PDE_{cGMP}=cGMP hydrolysing PDE
PDEi=inhibitor of a PDE (also known as I:PDE)
PDE5=phosphodiesterase type 5
PDE5i=inhibitor of PDE5
kDa=kilodalton
bp=base pair
kb=kilobase pair

### LIST OF SEQUENCES

### Which Forms Part of the Description

SEQ ID NO: 1

MEGALAANWSAEAANASAAPPGAEGNRTAGPPRRNEALARVEVAVLCLIL
LLALSGNACVLLALRTTRQKHSRLFFFMKHLSIADLVVAVFQVLPQLLWD
ITFRFYGPDLLCRLVKYLQVVGMFASTYLLLLMSLDRCLAICQPLRSLRR
RTDRLAVLATWLGCLVASAPQVHIFSLREVADGVFDCWAVFIQPWGPKAY
ITWITLAVYIVPVIVLATCYGLISFKIWQNLRLKTAAAAAAEAPEGAAAG
DGGRVALARVSSVKLISKAKIRTVKMTFIIVLAFIVCWTPFFFVQMWSVW
DANAPKEASAFIIVMLLASLNSCCNPWIYMLFTGHLFHELVQRFLCCSAS
YLKGRRLGETSASKKSNSSSFVLSHRSSSQRSCSQPSTA

SEQ ID NO: 2

MRLSAGPDAGPSGNSSPWWPLATGAGNTSREAEALGEGNGPPRDVRNEEL
AKLEIAVLAVTFAVAVLGNSSVLLALHRTPRKTSRMHLFIRHLSLADLAV
AFFQVLPQMCWDITYRFRGPDWLCRVVKHLQVFGMFASAYMLVVMTADRY
IAVCHPLKTLQQPARRSRLMIAAAWVLSFVLSTPQYFVFSMIEVNNVTKA
RDCWATFIQPWGSRAYVTWMTGGIFVAPVVILGTCYGFICYNIWCNVRGK
TASRQSKGAEQAGVAFQKGFLLAPCVSSVKSISRAKIRTVKMTFVIVTAY
IVCWAPFFIIQMWSVWDPMSVWTESENPTITITALLGSLNSCCNPWIYMF
FSGHLLQDCVQSFPCCQNMKEKFNKEDTDSMSRRQTFYSNNRSPTNSTGM
WKDSPKSSKSIKFIPVST

1. A pharmaceutical composition comprising a selective oxytocin antagonist admixed with a pharmaceutically acceptable carrier, diluent or excipient.

2. (canceled)

3. The pharmaceutical composition according to claim 1 wherein the-selective oxytocin antagonist is at least 20-fold selective for an oxytocin receptor as compared with a vasopressin receptor.

4.-6. (canceled)

7. A method of treating or preventing a male ejaculatory disorder in a human or animal which method comprises administering to a human or animal in need thereof an effective amount of a selective oxytocin antagonist; wherein said selective oxytocin antagonist is at least 20-fold selective for an oxytocin receptor as compared with a vasopressin receptor.

8. The method according to claim 7 wherein the male ejaculatory disorder is premature ejaculation.

9.-27. (canceled)

28. A method of treating or preventing a male ejaculatory disorder in a human or animal which method comprises administering to a human or animal in need thereof a pharmaceutical composition comprising an effective amount of a selective oxytocin antagonist, wherein the selective oxytocin antagonist is at least 20-fold selective for an oxytocin receptor as compared with a vasopressin receptor; and one or more PDE 5 inhibitors admixed with a pharmaceutically acceptable carrier, diluent or excipient.

29.-30. (canceled)

31. The method according to claim 7, wherein the selective oxytocin antagonist is admixed with a pharmaceutically acceptable carrier, diluent or excipient.

32. The method according to claim 8, wherein administration of the selective oxytocin causes an increase in latency to ejaculation in the human or animal.

33. The method according to claim 8, wherein administration of the selective oxytocin antagonist does not adversely affect penile erection in the human or animal.

34. The method according to claim 8, wherein administration of the selective oxytocin antagonist enhances endogenous penile erection in the human or animal.

35. The method according to claim 7, wherein the selective oxytocin antagonist is supplied in a pharmaceutical pack comprising one or more compartments wherein at least one compartment comprises one or more of a selective oxytocin antagonist.

36. The method according to claim 7, wherein the selective oxytocin antagonist comprises a compound having the structure 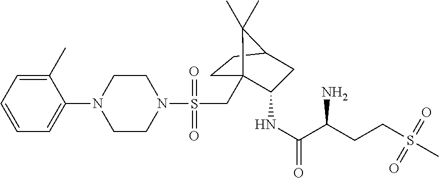 [SMILES: Cc1ccccc1N1CCN(S(=O)(=O)CC23CCC(C[C@@H]2NC(=O)[C@@H](N)CCS(C)(=O)=O)C3(C)C)CC1]
